(19) United States
(12) Patent Application Publication
(10) Pub. No.: US 2023/0157580 A1
(43) Pub. Date: May 25, 2023

(54) SYSTEM AND METHOD FOR DETERMINING CYCLING POWER

(71) Applicant: Orpyx Medical Technologies Inc., Calgary (CA)

(72) Inventors: SAMUEL CARL WILLIAM BLADES, VICTORIA (CA); ERIC CHRISTIAN HONERT, CALGARY (CA); BENNO MAURUS NIGG, CALGARY (CA); SADHIQ HUSSAIN NAZEER, CALGARY (CA)

(21) Appl. No.: 17/989,914

(22) Filed: Nov. 18, 2022

Related U.S. Application Data
(60) Provisional application No. 63/282,234, filed on Nov. 23, 2021.
(60) Provisional application No. 63/291,994, filed on Dec. 21, 2021.

(30) Foreign Application Priority Data
Sep. 27, 2022 (CA) 3176040

(51) Int. Cl. A61B 5/11; G16H 20/30

(52) U.S. Cl. CPC A61B 5/11; G16H 20/30; A61B 2562/0219

(57) ABSTRACT

A system, method and computer program product for determining cycling power. A plurality of force sensor readings are acquired from force sensors positioned underfoot. At least one revolution is identified. Force values are determined for the at least one revolution using aggregate force data. The cycling cadence, foot velocity, and foot angle are determined for each revolution. User mass is also determined. The cycling power associated with the force sensor readings is determined by inputting the force values, the cycling cadence, the foot velocity, and the foot angle, and the user mass to a machine learning model trained to predict the cycling power. The cycling power can then be provided to the user as feedback or stored for purposes such as later review and analysis. The inputs to the machine learning model can be determined entirely based off of sensor data received from a wearable device worn by the user.

### CROSS-REFERENCE TO RELATED APPLICATIONS

This application claims the benefit of priority of Canadian Patent Application No. 3,176,040 filed Sep. 27, 2022, U.S. Provisional Application No. 63/291,994 filed Dec. 21, 2021, and U.S. Provisional Application No. 63/282,234 filed Nov. 23, 2021. All of the above applications are incorporated herein by reference.

### FIELD

This document relates to systems and methods for processing data from sensors monitoring human movement or human activity. In particular, this document relates to determining cycling power using force sensor data.

### BACKGROUND

U.S. Pat. No. 10,004,946 (Ross) discloses a sensor system and method for monitoring power exerted by a person riding a bicycle. The sensory system has a pair of sensor insoles and a portable electronic device. The sensor insoles each have a plurality of force sensors, an accelerometer, and a transmitter for transmitting data from the plurality of force sensors and the accelerometer. A power measurement program operably installed in the computer memory of the portable electronic device receives the data and calculates power and other outputs based upon the data received.

US Pat. No. 7,599,806 (Hauschildt) discloses a power meter for a pedal and crank arm based drive mechanism, which measures and displays athletic performance information as a measure of a physical quantity, power, using a flexible force sensor inserted into a cycling shoe. Mathematical cycling models and data from the flexible force sensor are used to calculate the power.

### SUMMARY

The following summary is intended to introduce the reader to various aspects of the detailed description, but not to define or delimit any invention.

Systems, methods and computer program products for determining cycling power are disclosed. More particularly, in some examples, force sensor data can be acquired from a plurality of force sensors positioned underfoot of a user performing a cycling activity. Using the force sensor data, force values can be determined for a plurality of revolutions. A corresponding cadence, foot velocity, and foot angle can also be determined for each revolution. The force values, cadence, foot velocity, and foot angle, along with the user's mass can be provided as inputs to a machine learning model trained to determine the mechanical cycling power output by the user. The mechanical cycling power can then be provided to the user as feedback or stored for purposes such as review and analysis.

In some examples, the inputs to the machine learning model can be determined entirely based off of sensor data received from a wearable device worn by the user. The wearable device can include a plurality of force sensors and an inertial measurement unit. For example, an insole can be provided with the plurality of force sensors and the inertial measurement unit. Sensor data from the force sensors and the inertial measurement unit can be used to determine the force values, cadence, foot velocity and foot angle. This may allow the user to perform cycling activities at various locations, and using different bicycles, while still tracking and monitoring their mechanical cycling power output.

According to some aspects, the present disclosure provides a method for determining cycling power using force sensor data from a plurality of force sensors positioned underfoot. The method includes: identifying at least one revolution associated with the plurality of force sensors; obtaining a plurality of force sensor readings from the plurality of force sensors during the at least one revolution; determining force values for the at least one revolution using aggregate data based on the sensor readings; determining a user mass associated with the plurality of force sensor readings; for each revolution, determining a cycling cadence, a foot velocity, and a foot angle; and determining a mechanical cycling power associated with the plurality of force sensor readings by inputting the force values, the user mass, the cycling cadence, the foot velocity and the foot angle to a machine learning model trained to predict the mechanical cycling power.

The plurality of force sensors can be disposed on a wearable device that is worn on a foot.

The wearable device can include a deformable material.

The deformable material can be a foam.

The wearable device can be an insole.

The wearable device can be a shoe.

The wearable device can be a compression-fit garment.

The wearable device can be a sock.

The wearable device can include an inertial measurement unit.

The method can include outputting an output dataset, which can include the mechanical cycling power and/or the cycling cadence.

The method can include using the output dataset as an input to a game.

The output dataset can be used to execute an action in the game.

A scaling factor can be applied to the output dataset in the game.

The scaling factor can be an integer.

The scaling factor can have a value of 1.

An avatar can be generated in the game with motion defined according to the output dataset.

The output dataset can be used to model the dynamics of virtual objects and surroundings with which a user interacts in the game.

A game score in the game can be calculated based on the output dataset.

A training goal can be generated based on the output dataset and/or the game score.

A percentage of progress towards achieving the training goal can be calculated based on the output dataset and/or the game score.

The training goal can be a range of mechanical cycle power values that the user must stay within for a predetermined period of time.

The training goal can be a range of cycling cadence values that the user must stay within for a predetermined period of time.

A technique quality of a user performing a movement can be calculated from the output dataset.

A task readiness score can be calculated from the output dataset and/or the technique quality.

A first user can be challenged to replicate the output dataset of a second user in the game.

The wearable device can include at least one vibrotactile motor.

The at least one vibrotactile motor can generate a haptic signal based on the output dataset.

An audio signal can be generated based on the output dataset.

A visual display can be generated based on the output dataset.

The visual display can be an output dataset vs. time graph.

The cycling cadence can be determined as an average cadence for the corresponding revolution.

The cycling cadence can be determined based on inertial measurement data received from at least one inertial measurement unit associated with the plurality of force sensors.

The cycling cadence can be determined by: receiving accelerometer data from the at least one inertial measurement unit; identifying a revolution period of the corresponding revolution by: identifying a plurality of threshold crossing pairs, each threshold crossing pair including a first threshold crossing point and a second threshold crossing point, wherein the first threshold crossing point is identified as a first point when the accelerometer data crosses a specified cadence threshold while decreasing, and wherein the second threshold crossing point is identified as a second point when the accelerometer data crosses the specified cadence threshold while increasing; for each threshold crossing pair, identifying a signal minimum from the accelerometer data at a signal minimum point located between the first point and the second point; identifying the revolution period as a period extending between the signal minimums of adjacent threshold crossing pairs; and identifying the cycling cadence for the corresponding revolution by converting the revolution periods to a cadence value.

The revolution period can be identified based on a z-direction accelerometer signal component in an upward-downward direction from the accelerometer data.

The foot angle can be determined by: receiving gyroscope data from at least one inertial measurement unit; and determining a foot angle by integrating the gyroscope data in a sagittal plane.

The foot velocity can include a first foot velocity value in a forward-rearward direction and a second foot velocity value in the upward-downward direction, and the first foot velocity value and the second foot velocity value can be provided as separate inputs to the machine learning model.

The first foot velocity value can be determined by: receiving accelerometer data from at least one inertial measurement unit; and determining a first foot velocity value by integrating the accelerometer data in the forward-rearward direction.

The second foot velocity value can be determined by: receiving accelerometer data from at least one inertial measurement unit; and determining a second foot velocity value by integrating the accelerometer data in the upward-downward direction.

The method can include calculating an estimated mechanical cycling power using the first foot velocity value, the second foot velocity value, and the force values; and determining the mechanical cycling power by inputting the estimated mechanical cycling power to the machine learning model along with the force values, the user mass, the cycling cadence, the foot velocity and the foot angle.

The estimated mechanical cycling power P can be calculated according to:

P=F_{Y}−v_{Y}+F_{Z}×v_{Z}

where v_{Y} represents the first foot velocity value, v_{Z} represents the second foot velocity value, F_{Y} represents a first directional force component in the forward-rearward direction determined from the force values, and F_{Z} represents a second directional force component in the upward-downward direction determined from the force values.

The plurality of force sensors can include force-sensing resistors and the force values can be determined based on a sum of individual sensor force values from the force sensor readings.

The machine learning model can be a neural network model.

The machine learning model can be trained using training data generated by collecting training sensor readings and a training mechanical cycling power calculated from pedals equipped with force sensors.

The mechanical cycling power can be determined as a peak mechanical cycling power for each revolution.

The mechanical cycling power can be determined as a time-continuous mechanical cycling power for each revolution.

According to some aspects, there is also provided a system for determining cycling power. The system comprises: a plurality of force sensors positionable underfoot; one or more processors communicatively coupled to the plurality of force sensors; and a non-transitory storage memory storing a machine learning model trained to predict mechanical cycling power; wherein the one or more processors are configured to: identify at least one revolution associated with the plurality of force sensors; obtain a plurality of force sensor readings from the plurality of force sensors during the at least one revolution; determine force values for the at least one revolution using aggregate data based on the force sensor readings; determine a user mass associated with the plurality of force sensor readings; for each revolution, determine a cycling cadence, a foot velocity, and a foot angle; and determine a mechanical cycling power associated with the plurality of force sensor readings by inputting the force values, the user mass, the cycling cadence, the foot velocity and the foot angle to a machine learning model trained to predict the mechanical cycling power.

The system can include a wearable device on which the plurality of force sensors are disposed, and the wearable device can be worn on a foot.

The wearable device can include a deformable material.

The deformable material can be a foam.

The wearable device can be an insole.

The wearable device can be a shoe.

The wearable device can be a compression-fit garment.

The wearable device can be a sock.

The one or more processors can be further configured to output an output dataset, and the output dataset can include the mechanical cycling power and/or the cycling cadence.

The one or more processors can be further configured to use the output dataset as an input to a game.

The one or more processors can be further configured to execute an action in the game based on the output dataset.

The one or more processors can be further configured to apply a scaling factor to the output dataset in the game.

The scaling factor can be an integer.

The scaling factor can have a value of 1.

The one or more processors can be further configured to generate an avatar in the game with motion defined according to the output dataset.

The one or more processors can be further configured to model the dynamics of virtual objects with which a user interacts in the game based on the output dataset.

The one or more processors can be further configured to compute a game score in the game based on the output dataset.

The one or more processors can be further configured to generate a training goal based on the output dataset and/or the game score.

The one or more processors can be further configured to calculate a percentage of progress towards achieving the training goal based on the output data and/or the game score.

The training goal can be a range of mechanical cycling power values to stay within for a predetermined period of time

The training goal can be a range of cycling cadence values to stay within for a predetermined period of time.

The one or more processors can be further configured to determine a technique quality of a user performing a movement based on the output dataset.

The one or more processors can be further configured to determine a task readiness score based on the output dataset and/or the technique quality.

The one or more processors can be further configured to challenge a first user to replicate the output dataset of a second user in the game.

The system can include at least one vibrotactile motor.

The at least one vibrotactile motor can be configured to generate a haptic signal based on the output dataset.

The one or more processors can be further configured to generate an audio signal based on the output dataset.

The one or more processors can be further configured to generate a visual display based on the output dataset.

The visual display can be an output dataset vs. time graph.

The one or more processors can be configured to determine the cycling cadence as an average cadence for the corresponding revolution.

The system can further include at least one inertial measurement unit associated with the plurality of force sensors. The at least one inertial measurement unit can be communicatively coupled to the one or more processors; and the one or more processors can be configured to determine the cycling cadence based on inertial measurement data received from the at least one inertial measurement unit.

The one or more processors can be configured to determine the cycling cadence by: receiving accelerometer data from the at least one inertial measurement unit; identifying a revolution period of the corresponding revolution; and identifying the cycling cadence for the corresponding revolution by converting the revolution periods to a cadence value. Identifying the revolution period of the corresponding revolution can be done by identifying a plurality of threshold crossing pairs, each threshold crossing pair including a first threshold crossing point and a second threshold crossing point, wherein the first threshold crossing point is identified as a first point when the accelerometer data crosses a specified cadence threshold while decreasing, and wherein the second threshold crossing point is identified as a second point when the accelerometer data crosses the specified cadence threshold while increasing; for each threshold crossing pair, identifying a signal minimum from the accelerometer data at a signal minimum point located between the first point and the second point; and identifying the revolution period as a period extending between the signal minimums of adjacent threshold crossing pairs.

The one or more processors can be configured to identify the revolution period based on a z-direction accelerometer signal component in an upward-downward direction from the accelerometer data.

The system can further include at least one inertial measurement unit associated with the plurality of force sensors. The at least one inertial measurement unit can be communicatively coupled to the one or more processors. The one or more processors can be configured to determine the foot angle by: receiving gyroscope data from at least one inertial measurement unit; and determining a foot angle by integrating the gyroscope data in a sagittal plane.

The one or more processors can be configured to determine the foot velocity to include a first foot velocity value in a forward-rearward direction and a second foot velocity value in the upward-downward direction, and to provide the first foot velocity value and the second foot velocity value as separate inputs to the machine learning model.

The one or more processors can be configured to determine the first foot velocity value by: receiving accelerometer data from at least one inertial measurement unit; and determining a first foot velocity value by integrating the accelerometer data in the forward-rearward direction.

The one or more processors can be configured to determine the second foot velocity value by: receiving accelerometer data from at least one inertial measurement unit; and determining a second foot velocity value by integrating the accelerometer data in the upward-downward direction.

The one or more processors can be configured to: calculate an estimated mechanical cycling power using the first foot velocity value, the second foot velocity value, and the force values; and determine the mechanical cycling power by inputting the estimated mechanical cycling power to the machine learning model along with the force values, the user mass, the cycling cadence, the foot velocity and the foot angle.

The one or more processors can be configured to calculate the estimated mechanical cycling power P according to:

P=F_{Y}−v_{Y}+F_{Z}×v_{Z}

where v_{Y} represents the first foot velocity value, v_{Z} represents the second foot velocity value, F_{Y} represents a first directional force component in the forward-rearward direction determined from the force values, and F_{Z} represents a second directional force component in the upward-downward direction determined from the force values.

The plurality of force sensors can include force-sensing resistors and the one or more processors can be configured to determine the force values based on a sum of individual sensor force values from the force sensor readings.

The machine learning model can be a neural network model.

The machine learning model can be trained using training data generated by collecting training sensor readings and a training mechanical cycling power calculated from pedals equipped with force sensors.

The machine learning model can be defined to determine the mechanical cycling power to include a peak mechanical cycling power for each revolution.

The machine learning model can be defined to determine the mechanical cycling power to include a time-continuous mechanical cycling power for each revolution.

According to some aspects, there is provided a non-transitory computer readable medium storing computer-executable instructions, which, when executed by a computer processor, cause the computer processor to carry out a method of determining cycling power. The method includes identifying at least one revolution associated with a plurality of force sensors positioned underfoot; obtaining a plurality of force sensor readings from the plurality of force sensors during the at least one revolution; determining force values for the at least one revolution using aggregate data based on the force sensor readings; determining a user mass associated with the plurality of force sensor readings; for each revolution, determining a cycling cadence, a foot velocity, and a foot angle; determining a mechanical cycling power associated with the plurality of force sensor readings by inputting the force values, the user mass, the cycling cadence, the foot velocity and the foot angle to a machine learning model trained to predict the mechanical cycling power; and outputting the mechanical cycling power.

The non-transitory computer readable medium can store computer-executable instructions, which, when executed by a computer processor, cause the computer processor to carry out the method of determining cycling power, where the method is described herein.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a block diagram illustrating an example of a system for determining cycling power;

FIG. 2 is a plan view of an example of a wearable device incorporating a sensing unit that can be used in the system of FIG. 1;

FIG. 3 is a flowchart illustrating an example of a method for determining cycling power;

FIG. 4 is a flowchart illustrating an example of a method for determining a cycling cadence that may be used with the method shown in FIG. 3;

FIG. 5 is a diagram illustrating a plot of the z-directional component of an acceleration signal received from an inertial measurement unit that may be used in the method of FIG. 4;

FIG. 6 is a diagram illustrating a plot of the estimated cycling cadence determined by an implementation of the method of FIG. 4 as compared to the cadence determined by instrumented force pedals;

FIG. 7 is a diagram illustrating a plot of the estimated peak cycling power determined by an implementation of the method of FIG. 3 using force sensor data from forces sensors mounted on an insole as compared to the peak cycling power determined by instrumented force pedals;

FIG. 8 is a diagram illustrating a plot of the estimated peak cycling power determined by a physics-based approach using force sensor data from forces sensors mounted on an insole as compared to the peak cycling power determined by instrumented force pedals.

### DETAILED DESCRIPTION

Various apparatuses or processes or compositions will be described below to provide an example of an embodiment of the claimed subject matter. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements. In addition, numerous specific details are set forth in order to provide a thorough understanding of the subject matter described herein. However, it will be understood by those of ordinary skill in the art that the subject matter described herein may be practiced without these specific details. In other instances, well-known methods, procedures and components have not been described in detail so as not to obscure the subject matter described herein. The description is not to be considered as limiting the scope of the subject matter described herein.

The terms “coupled” or “coupling” as used herein can have several different meanings depending in the context in which these terms are used. For example, the terms coupled or coupling can have a mechanical, electrical or communicative connotation. For example, as used herein, the terms coupled or coupling can indicate that two elements or devices are directly connected to one another or connected to one another through one or more intermediate elements or devices via an electrical element, electrical signal, or a mechanical element depending on the particular context. Furthermore, the term “communicative coupling” may be used to indicate that an element or device can electrically, optically, or wirelessly send data to another element or device as well as receive data from another element or device.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof. Furthermore, the wording “at least one of A and B” is intended to mean only A (i.e. one or multiple of A), only B (i.e. one or multiple of B), or a combination of one or more of A and one or more of B.

Terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

Described herein are systems, methods and devices for determining the cycling power of a user while cycling. Determining a user's cycling power can provide insightful movement and activity data. Knowledge of cycling power may be useful for various cycling applications, such as athletic training, occupational training (e.g. cycling training for police), power generation (e.g. cycling on bicycles that convert mechanical energy into electrical energy) and recreational cycling.

Cycling power can provide valuable biometric feedback to users who are using a stationary or real bicycle. Feedback on the user's cycling power may allow the user to continue performing at their maximum capacity for an extended period without burning out.

For example, an athlete cycling uphill may wish to cycle at a speed that maintains their cycling power from when they were on flat ground. If the user cycles uphill at a speed that causes them to exceed the flat ground cycling power, they may tire faster and may not be able to perform at their maximum capacity for the same extended period.

Feedback on the user's cycling power may allow the user to optimize their training for the maximum training effect. For example, cycling power provides the user with instantaneous quantification of their work rate, which can be used in specific training techniques such as interval training.

Feedback on the user's cycling power can also be integrated into third party connected fitness apps to allow the user to engage with these systems.

Cycling power output is also related to a user's maximum oxygen intake. The greater the oxygen intake, the more oxygen that may be delivered to the muscles, which may contribute to a greater cycling power. Conversely, determining the user's peak cycling power may provide an indication of the user's oxygen intake.

The systems, methods and devices described herein can determine a user's mechanical cycling power using a plurality of sensors positioned underfoot. The systems, methods, and devices can use sensors attached to, or contained within, wearable devices or cycling equipment to measure and monitor data relating to movement or activity of a user. The measured data from the sensors can be used to calculate the mechanical cycling power.

The sensors can be force sensors and can be provided in the insole of a shoe or within the footwear worn by the user. As used herein, the term “force” is used broadly and can refer to raw force (i.e. with units of N), or pressure resulting from a raw force (i.e. with units of N/m²). The force data acquired by the force sensors can be used to determine the level of force applied by a user's foot while cycling. This force data can be used to derive additional force derivatives or force-based metrics, such as the force output, mean force or the peak force for the user or activity. The force data, and other data derived therefrom, can be used to determine the mechanical cycling power generated by the user that may be useful for medical, fitness, athletic, security, gaming, entertainment or other purposes.

The systems, methods, and devices described herein may be implemented as a combination of hardware or software. In some cases, the systems, methods, and devices described herein may be implemented, at least in part, by using one or more computer programs, executing on one or more programmable devices including at least one processing element, and a data storage element (including volatile and non-volatile memory and/or storage elements). These devices may also have at least one input device (e.g. a pushbutton keyboard, mouse, a touchscreen, and the like), and at least one output device (e.g. a display screen, a printer, a wireless radio, and the like) depending on the nature of the device.

Some elements that are used to implement at least part of the systems, methods, and devices described herein may be implemented via software that is written in a high-level procedural language such as object oriented programming. Accordingly, the program code may be written in any suitable programming language such as Python or C for example. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, magnetic disk, optical disc) or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods described herein may be capable of being distributed in a computer program product including a computer readable medium that bears computer usable instructions for one or more processors. The medium may be provided in various forms, including non-transitory forms such as, but not limited to, one or more diskettes, compact disks, tapes, chips, and magnetic and electronic storage. Alternatively, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

The present disclosure relates in general to a system, method and device that can be used to determine mechanical cycling power for a user cycling on a stationary bicycle or a real bicycle. The systems, methods and devices described herein can be used to determine mechanical cycling power using force sensor data from a plurality of force sensors positioned underfoot. The force sensor data can be used to determine force values for a plurality of revolutions. The term “revolution” generally refers to one complete (360°) rotation of a cycling pedal (e.g. when the right pedal starts at its highest point, rotates to the lowest point, then rises back up to its highest point).

Directly measuring the force applied by a user using underfoot force sensors (as opposed to deriving the force data from other wearable sensors such as accelerometers) can contribute to more accurate power calculations.

For each revolution, a corresponding cadence, foot velocity and foot angle can be calculated. The force values, cadence, foot velocity and foot angle can be input to a machine learning model along with the user's mass. The machine learning model can be trained to output an estimate of mechanical cycling power in response to receiving the mass, force values, cadence, foot velocity and foot angle as inputs. The output from the machine learning model can be stored for review or analysis and/or provided to the user as dynamic feedback on their cycling power output.

The systems, methods and devices described herein can also include one or more inertial measurement units (IMUs). Each IMU can be associated with a corresponding plurality of force sensors. That is, each IMU can be configured to collect inertial measurement data relating to movement of the same foot under which the force sensors are positioned.

IMU data from the one or more IMUs can be used to determine one or more inputs to the machine learning model. For example, the cadence and/or the foot velocity and/or the foot angle may be determined wholly or partially using IMU data. These IMU-derived inputs can be provided to the machine learning model in conjunction with the force sensor derived inputs in order to determine the mechanical cycling power for the user.

The systems, methods and devices described herein can be used to determine mechanical cycling power for a single foot based on sensor data acquired for that foot. The machine learning model can be trained to determine the mechanical cycling power for a single foot based on inputs relating to one or more revolutions for that foot.

Determining the mechanical cycling power for both feet of a user performing an activity can include providing inputs to the machine learning model for each foot. The inputs provided to the machine learning model can be determined based on sensor data acquired individually for each foot.

Determining the mechanical cycling power for both feet can also allow further metrics to be determined. For example, a bilateral power asymmetry can be calculated by comparing the mechanical cycling power determined for the left foot during a revolution to the mechanical cycling power determined for the right foot during its subsequent revolution (or vice versa).

The systems, methods and devices described herein can also be used to acquire sensor data for both of a user's feet at the same time. This generally requires a separate plurality of force sensors for each foot (e.g. where the force sensors are incorporated into a wearable device or mounted on a pedal). Where an IMU is included, a separate IMU can be provided for each foot. This allows the IMU to collect inertial measurement data relating to movement of that foot. The inertial measurement data specific to the foot can then be used to determine inputs to the machine learning model for the individual revolutions of that foot.

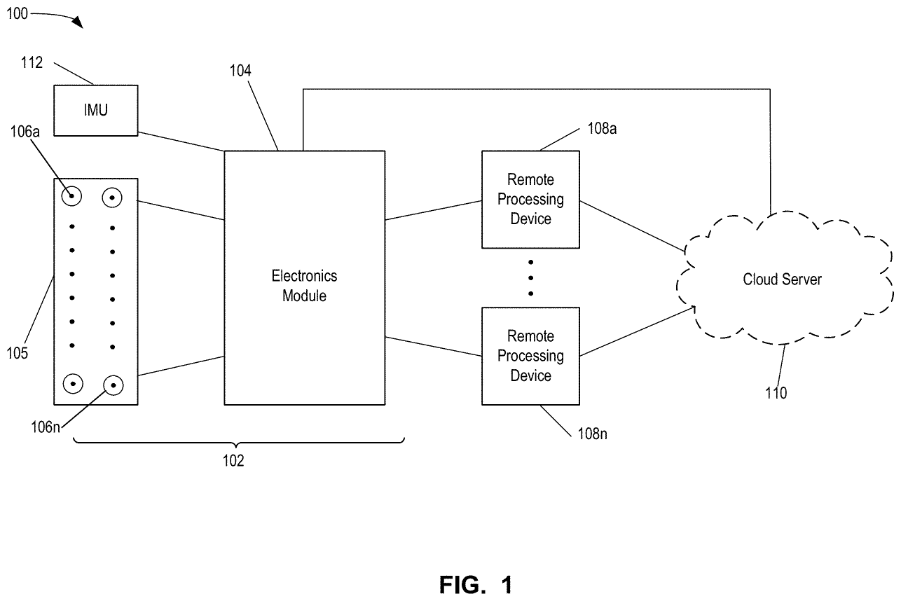

Referring now to FIG. 1, shown therein is a block diagram illustrating an example system 100 that can be used to determine mechanical cycling power for a user. System 100 includes a plurality of sensors positioned underfoot of a user performing a cycling activity. The sensors may be provided using a wearable device and/or cycling equipment.

System 100 includes an input unit 102 (also referred to herein as an input device), one or more processing devices 108 (also referred to herein as a receiving device or an output device) and an optional remote cloud server 110. As will be described in further detail below, the input unit 102 may, for example, be combined with or integrated into a carrier unit such as a wearable device or a piece of cycling equipment.

Input unit 102 generally includes a sensing unit 105. The sensing unit 105 can include a plurality of sensors 106a -106n . The plurality of sensors 106a -106n can be configured to collect force data from underneath a user's foot.

In the example shown, input unit 102 also includes an inertial measurement unit (IMU) 112. IMU 112 can include one or more sensors for measuring the position and/or motion of the user's foot (e.g. via a carrier unit). For example, IMU 112 may include sensors such as a gyroscope, accelerometer (e.g., a three-axis accelerometer), magnetometer, orientation sensors (for measuring orientation and/or changes in orientation), angular velocity sensors, or inclination sensors. Generally, IMU 112 includes at least an accelerometer. The IMU 112 also typically includes a gyroscope.

The IMU 112 can also be positioned underneath a user's foot. However, the IMU 112 need not be positioned underfoot so long as the IMU 112 can collect inertial measurement data relating to the position and/or motion of the foot.

The carrier unit can be configured to position the sensors 106 in contact with (or close proximity to) a user's body to allow the sensors 106 to measure an aspect of the cycling activity being performed by the user. The plurality of sensors 106a -106n may be configured to measure a particular sensed variable at a location of a user's body when the carrier unit is engaged with the user's body (e.g. when the user is wearing a wearable device containing the sensors 106 or when the user is using fitness equipment containing the sensors 106). In system 100, the plurality of sensors 106a -106n can be arranged to measure force underneath the foot (underfoot) of a user.

In some examples, the carrier unit may include one or more wearable devices. The wearable devices can be manufactured of various materials such as fabric, doth, polymer, or foam materials suitable for being worn close to, or in contact with, a user's skin. All or a portion of the wearable device may be made of breathable materials to increase comfort while a user is performing an activity.

In some examples, the wearable device may be formed into a garment or form of apparel such as a sock, a shoe, and an insole. Some wearable devices such as socks may be in direct contact with a user's skin. Some wearable devices, such as shoes, may not be in direct contact with a user's skin but still positioned within sufficient proximity to a user's body to allow the sensors to acquire the desired readings.

In some cases, the wearable device may be a compression-fit garment. The compression-fit garment may be manufactured from a material that is compressive. A compression-fit garment may minimize the impact from “motion artifacts” by reducing the relative movement of the wearable device with respect to a target location on the user's body. In some cases, the wearable device may also include anti-slip components on the skin-facing surface. For example, a silicone grip may be provided on the skin-facing surface of the wearable device to further reduce the potential for motion artifacts.

The wearable device can be worn on a foot. For example, the wearable device may be a shoe, a sock, or an insole, or a portion of a shoe, a sock, or an insole. The wearable device may include a deformable material, such as foam. This may be particularly useful where the wearable device is a shoe or insole.

The plurality of sensors 106a -106n can be positioned to acquire sensor readings from specified locations on a user's body (via the arrangement of the sensors on the carrier unit). The sensors 106 can be integrated into the material of the carrier unit (e.g. integrated into a wearable device or fitness equipment). Alternatively, the sensors 106 can be affixed or attached to the carrier unit, e.g. printed, glued, laminated or ironed onto a surface, or between layers, of a wearable device or fitness equipment.

In some examples, the carrier unit may include cycling equipment. The cycling equipment may include components of a bicycle on which a user can exert force with their foot while performing a cycling activity. For example, the carrier unit may be provided by the pedals of a bicycle.

In some examples, the sensors 106 and IMU 112 may be provided by the same carrier unit. Alternatively, the IMU 112 may be provided by a separate carrier unit.

For clarity, the below description relates to a carrier unit in the form of an insole. The insole carrier unit may be provided in various forms, such as an insert for footwear or integrated into a shoe. However, other carrier units may be implemented using the systems and methods described herein, such as the wearable devices and cycling equipment described above. Incorporating the sensing unit 105 (and optionally the IMU 112) into a carrier unit in the form of a wearable device may be desirable as it allows cycling power to be determined for a user at various locations and without requiring specifically configured cycling equipment.

The below description relates to an insole in which the plurality of sensors 106 are force sensors. Various types of force sensors may be used, such as force sensing resistors (also referred to as “sensels” or sensing elements), pressure sensors, piezoelectric tactile sensors, elasto-resistive sensors, capacitive sensors or more generally any type of force sensor that can be integrated into a wearable device or fitness equipment capable of collecting force data underfoot.

The plurality of sensors 106 may be arranged into a sensor array. A sensor array (as used herein) refers to a series of sensors arranged in a defined grid. The plurality of sensors 106 can be arranged in various types of sensor arrays.

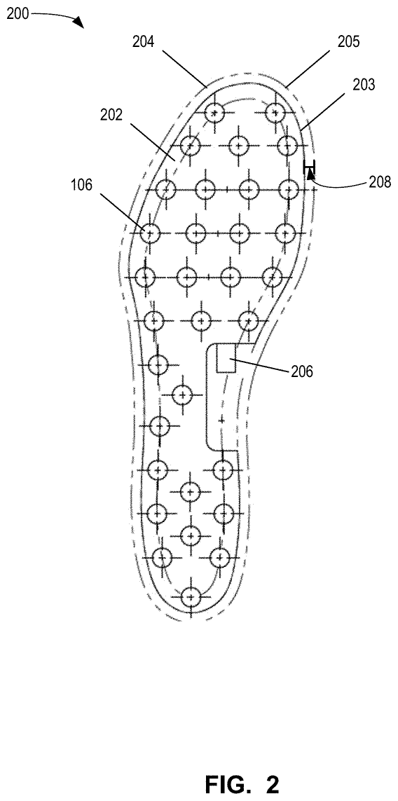

For example, the plurality of sensors 106 can be provided as a set of discrete sensors (see e.g. FIG. 2). A discrete sensor is an individual sensor that acquires a sensor reading at a single location. A set of discrete sensors generally refers to multiple discrete sensors that are arranged in a spaced apart relationship in a sensing unit.

Sensors 106a -106n may be arranged in a sparse array of discrete sensors that includes void locations where no sensors 106 are located. Alternatively, sensors 106a -106n may be arranged in a continuous or dense sensor array in which sensors 106 are arranged in a continuous, or substantially continuous manner, across the grid.

Discrete sensors can provide an inexpensive alternative to dense sensor arrays for many applications. However, because no sensors are positioned in the interstitial locations between the discrete sensors and the void locations external to the set of discrete sensors, no actual sensors readings can be acquired for these locations. Accordingly, depending on the desired resolution for the force sensor data, sensor readings may be estimated (rather than measured) at the interstitial locations and at the void locations external to the set of discrete sensors in order to provide sensor data with similar resolution to a dense sensor array. Alternatively, where lower resolution force sensor data is sufficient, sensor readings may not be estimated.

Various interpolation and extrapolation techniques may be used to estimate sensor values at interstitial locations and external void locations. In some cases, sensor values may be estimated using methods for synthesizing sensor data.

According to some aspects, a method for synthesizing sensor data can include: obtaining a plurality of sensor readings from a corresponding plurality of sensors, the plurality of sensors arranged in a first predetermined pattern, wherein the first predetermined pattern maps each of the plurality of sensors to respective locations on a wearable device; and based on the plurality of sensor readings and a plurality of estimation weights, estimating a plurality of synthesized sensor readings for a corresponding plurality of synthesized sensors, the plurality of synthesized sensors arranged in a second predetermined pattern, wherein the second predetermined pattern maps each of the plurality of synthesized sensors to respective locations on the wearable device.

The plurality of estimation weights can be predetermined in a preprocessing phase, and the preprocessing phase can include: obtaining training data, the training data including a plurality of sets of physical sensor readings from physical sensors arranged according to both the first and second predetermined patterns; filtering the training data to obtain filtered training data; using the filtered training data, computing an average sensor reading for each physical sensor to produce an input data set and a reference data set, the input data set including average sensor readings for sensors corresponding to the first predetermined pattern, the reference data set including average sensor readings for sensors corresponding to the second predetermined pattern; and optimizing the estimation weights.

Optimizing the estimation weights can include: initially estimating the estimation weights; computing estimated sensor values based on the input data set and the estimation weights; and performing gradient descent optimization to update the estimation weights, where the gradient descent optimization compares error between the estimated sensor values and the reference data set.

Filtering the training data can include resizing each instance of the training data to a common size. Filtering the training data can also include: dividing the training data into stance data and swing data; and resizing each instance in the set of stance data to a common size.

The plurality of sensor readings can be associated with an activity, and the plurality of synthesized sensor readings can be estimated when the activity is an activity requiring more sensors than can be provided by the plurality of sensors in the first predetermined pattern. The activity can be running, jogging, walking, or cycling.

The method can include predetermining and optimizing estimation weights associated with a specific activity.

The first predetermined pattern can include at least 32 locations.

The first predetermined pattern can include sensors arranged in a 2-3-4-4-4-3 arrangement in a forefoot portion. The first predetermined pattern can include sensors arranged in a 1-1-1 arrangement in a midfoot portion. The first predetermined pattern can include sensors arranged in a 2-1-2-1-2-1 arrangement in a heel portion.

The second predetermined pattern can include at least 32 locations. The second predetermined pattern can include at least 68 locations.

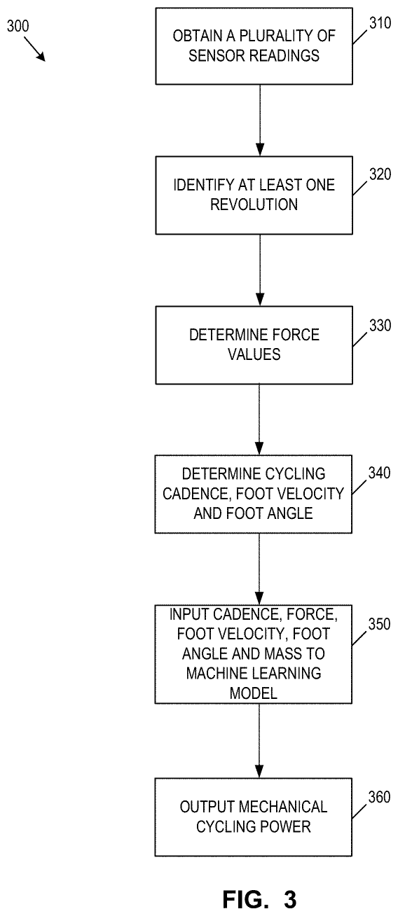

System 100 can be configured to implement a method of determining mechanical cycling power. The method of determining mechanical cycling power may be implemented using a processor/controller of the input device 102, a remote processing device 108, or cloud server 110. An example method 300 for determining cycling power is shown in FIG. 3 and described in further detail herein below.

As shown in FIG. 1, input unit 102 includes an electronics module 104 coupled to the plurality of sensors 106 and to optional IMU 112. In some cases, the electronics module 104 can include a power supply, a controller or other processing unit, a memory, a signal acquisition unit operatively coupled to the controller and to the plurality of sensors 106 (and to IMU 112), and a wireless communication module operatively coupled to the controller.

Generally, the sensing unit refers to the plurality of sensors 106 and the signal acquisition unit. The signal acquisition unit may provide initial analog processing of signals acquired using the sensors 106, such as amplification. The signal acquisition unit may also include an analog-to-digital converter to convert the acquired signals from the continuous time domain to a discrete time domain. The analog-to-digital converter may then provide the digitized data to the controller for further analysis or for communication to a remote processing device 108 or remote cloud server 110 for further analysis.

Optionally, the electronics module 104 may include a controller or other processing device configured to perform the signal processing and analysis. In such cases, the controller on the electronics module 104 may be configured to process the received sensor readings in order to determine cycling power. In some cases, the controller may be coupled to the communication module (and thereby the sensing unit) using a wired connection such as Universal Serial Bus (USB) or other port.

The electronics module 104 can be communicatively coupled to one or more remote processing devices 108a -108n , e.g. using a wireless communication module (e.g., Bluetooth, Bluetooth Low-Energy, WiFi, ANT+ IEEE 802.11, etc.). The remote processing devices 108 can be any type of processing device such as a personal computer, a tablet, and a mobile device (such as a smartphone, a smartwatch or a wristband) for example. The electronics modules 104 can also be communicatively coupled to a remote cloud server 110 over, for example, a wide area network such as the Internet.

Each remote processing device 108 and optional remote cloud server 110 typically includes a processing unit, an output device (such as a display, speaker, and/or tactile feedback device), a user interface, an interface unit for communicating with other devices, Input/Output (I/O) hardware, a wireless unit (e.g. a radio that communicates using CDMA, GSM, GPRS or Bluetooth protocol according to standards such as IEEE 802.11a, 802.11b, 802.11g, or 802.11n), a power unit and a memory unit. The memory unit can include RAM, ROM, one or more hard drives, one or more flash drives, or some other suitable data storage elements such as disk drives, etc.

The processing unit controls the operation of the remote processing device 108 or the remote cloud server 110 and can be any suitable processor, controller or digital signal processor that can provide sufficient processing power processor depending on the desired configuration, purposes and requirements of the system 100.

The display can be any suitable display that provides visual information. For instance, the display can be a cathode ray tube, a flat-screen monitor and the like if the remote processing device 108 or remote cloud server 110 is a desktop computer. In other cases, the display can be a display suitable for a laptop, tablet or handheld device such as an LED or LCD-based display and the like.

System 100 can generally be used for determining cycling power based on sensor readings received from a plurality of sensors positioned underfoot. In some cases, system 100 may also track additional data derived from the sensor readings. The sensor readings, cycling power data, and derived data may be monitored, stored, and analyzed for the user. Aspects of the monitoring, storage and analysis of biometric features and other metrics may be performed by one or more of the input unit 102, and/or a remote processing device 108, and/or the cloud server 110. For example, a non-transitory storage memory of one or more of the input unit 102, and/or a remote processing device 108, and/or the cloud server 110 can store a machine learning model trained to predict mechanical cycling power.

A remote cloud server 110 may provide additional processing resources not available on the input unit 102 or the remote processing device 108. For example, some aspects of processing the sensor readings acquired by the sensors 106 may be delegated to the cloud server 110 to conserve power resources on the input unit 102 or remote processing device 108. In some cases, the cloud server 110, input unit 102 and remote processing device 108 may communicate in real-time to provide timely feedback to a user regarding the sensor readings, cycling power data, and other related data such as cadence.

In the example system 100 illustrated in FIG. 1, a single input unit 102 is shown. However, system 100 may include multiple input units 102 associated with the same user. For example, system 100 may include two separate input units 102, each input unit 102 associated with one of the user's legs. Sensor data from an individual input unit 102 may be used to determine the mechanical cycling power for the user's corresponding leg.

Accordingly, the system 100 may include a separate sensing unit 105 and a separate IMU 112 for each foot of a user. This may allow the mechanical cycling power to be determined separately for each of the user's feet.

Each IMU 112 is associated with a single foot. Accordingly, separate IMUs 112 can be provided for each foot.

Certain hardware and software features of the system may be enabled, disabled, or changed. For example, a user may enable or disable certain sensors 106, which may be desirable if the user has a foot condition which inhibits them from activating certain sensors, such as a broken or missing toe. Sampling rate may also be modifiable. Sampling rate may be modified to minimize processing time and to save memory, or to increase data output to gain deeper insights. The location of processing (the input unit 102, the remote processing device 108, or the cloud server 110) may also be changed. If additional sensors are included in the wearable device (e.g. the IMU 112, temperature sensors, and/or GPS), certain sensor types may be enabled or disabled. For example, a GPS system can be disabled to conserve battery power of a carrier unit, if a user operates the carrier unit while cycling on a stationary bicycle at home.

Referring now to FIG. 2, shown therein is an example of an insole 200 that includes a sensing unit 202. The insole 200 is an example of an input device 102 that may be used in the system 100 shown in FIG. 1. The insole 200 may be the footwear insert described in PCT Application No. PCT/CA2020/051520 published on May 20, 2021, which is incorporated herein by reference. The insole 200 may be an Orpyx SI® Sensory Insole sold by Orpyx Medical Technologies Inc.

The insole 200 includes a sensor unit 202 and an optional liner 204. The liner 204 can provide a protective surface between the sensor unit 202 and a user's foot. The liner 204 may have a slightly larger profile as compared to the sensor unit 202. That is, the outer perimeter 203 of the sensor unit 202 may be inwardly spaced from the outer perimeter 205 of the liner 204 by an offset 208. The offset 208 may be substantially consistent throughout the perimeter of the sensor unit 202 such that the sensor unit 202 is completed covered by the liner 204.

Optionally, the sensor unit 202 can include an IMU (not shown), The sensor unit 202 can also include a connector 206. The connector 206 may provide a coupling interface between the plurality of sensors 106 (and the optional IMU) and an electronics module (not shown) such as electronics module 104. The coupling interface can allow signals from the sensors 106 and/or IMU to be transmitted to the electronics module. In some cases, the coupling interface may also provide control or sampling signals from the electronics module to the sensors 106 and/or IMU.

Optionally, the sensor unit 202 can also include one or more temperature sensors (not shown) and/or a GPS system (not shown),

The arrangement of sensors 106 in the sensor unit 202 is an example of a sparse sensor array that may be used to collect force sensor data. The arrangement of sensors 106 illustrated in FIG. 2 is an example, and various different types of force sensors, force sensor arrays, and arrangements of force sensors may be used. For example, sensor units containing a dense force sensor array (e.g. a Pedar® insole or Tekscan® system) may also be used,

Incorporating the sensor unit 202 in a wearable device such as insole 200 may provide a number of advantages. Pedal or crank-mounted sensing systems may require specialized installation for a given bicycle, By contrast, including the sensor unit 202 in a wearable device (e.g. an insole 200) does not require any special installation or modifications to a bicycle. This also allows a user to easily measure cycling power on multiple bicycles without any additional installation or removal of sensing systems.

Incorporating the sensing system into a wearable device may help reduce the cost of the sensing system. Furthermore, the wearable device may allow a user to measure force and other sensor data (e.g. IMU sensor data) while performing activities other than just cycling (e.g. running, walking, jumping, gaming etc.). This can further offset the cost of the sensing system, as a single sensing system may be used for multiple activities, rather than requiring separate specialized sensing systems for cycling and for other activities.

Referring now to FIG. 3, shown therein is an example method 300 for determining cycling power using force sensor data from a plurality of force sensors positioned underfoot. The method 300 may be used with a plurality of sensors configured to measure human movement or human activity, such as sensors 106. Method 300 is an example of a method for determining cycling power in which a machine learning model is used to determine the cycling power based on sensor readings acquired from the plurality of force sensors.

In the example of method 300, cycling power is determined using a machine learning model configured to receive a plurality of model inputs. Many existing approaches may calculate cycling power using simple equations of motion, such as:

$$P = T * ω = F * sin θ * r * ω \qquad ( 1 )$$

$$P = \frac{W}{t} = \frac{F * d}{t} = F * v \qquad ( 2 )$$

where P represents power, T represents torque, ω represents angular velocity, F represents force applied to the pedals, θ represents foot angle (the angle of the foot relative to the horizontal axis of the bicycle—i.e. the axis extending along the forward-rearward direction of the bike), r represents crank arm length, d represents distance, t represents time, and v represents velocity.

As shown in the example above, many existing approaches to determining cycling power require knowledge of the crank arm length. This typically requires a user to manually determine and input the crank arm length. This may be particularly challenging for users when switching between different bikes which can have different crank arm lengths.

By contrast, the example method 300 can determine the cycling power absent knowledge of the crank arm length. This can provide a more user-friendly method of determining the cycling power that is particularly useful for users who switch between bikes.

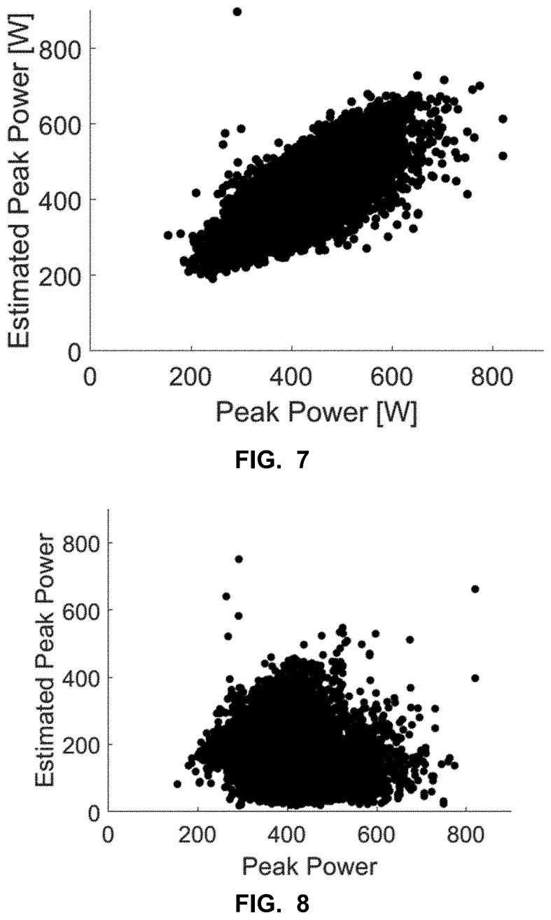

As shown in FIGS. 7 and 8 (described herein below), implementations of method 300 have been found to estimate mechanical cycling power with higher accuracy than calculations made with standard physics equations, such as equations (1) and (2) identified above.

In method 300, the inputs to the machine learning model include a plurality of preprocessed inputs determined based on sensor data from force sensors and one or more inertial measurement units. The model inputs also include a mass of the user performing a cycling activity. The mass is thus associated with the user generating the sensor readings captured by the force sensors and inertial measurement units.

The user mass may be determined in various ways. Typically, the user mass can be determined prior to the cycling activity. For example, user mass may be measured using the force sensors underfoot of a user. Alternatively, mass may be measured using a separate mass-measurement device such as a scale. In some cases, mass may be input manually, e.g. by a user interacting with an application on processing device 108. In some cases, determining the user mass may require converting the user's measured weight to a mass value.

At 310, a plurality of sensor readings can be obtained from a corresponding plurality of sensors. The plurality of sensors can include a plurality of force sensors positioned underfoot (i.e. underneath the foot) of a user performing a physical activity. The plurality of force sensors can be configured to acquire force sensor data during at least one revolution of a user's foot while cycling.

The force sensors can be positioned at specified locations on a carrier unit such as a wearable device or a pedal. The force sensors can be configured to measure force data relating to human activity. As shown in FIG. 2, the plurality of sensors may be force sensors provided at various locations of an insole. The force sensors can measure force applied to the insole during cycling activities.

The plurality of sensors may also include one or more IMUs. Accordingly, the plurality of sensor readings acquired at 310 can include IMU sensor data received from the one or more IMUs.

Each IMU can be associated with the plurality of force sensors. For example, the IMU may be incorporated into the same wearable device as the plurality of force sensors. More generally, the IMU can be configured to collect IMU sensor data about a single foot of a user. This IMU sensor data can be acquired for the same foot for which the sensor readings were obtained from the plurality of force sensors.

The sensor readings acquired at 310 may be acquired as a time-continuous set of sensor readings. This may provide a time-continuous set of sensor data that can be used to determine the mechanical cycling power as a time-continuous cycling power and/or a peak power value for each revolution. Depending on the nature of the sensors and the signal preprocessing performed, the time-continuous sensor data may be discretized e.g. using an analog to digital conversion process. Even where the sensor data is discretized, the set of sensor data may allow the mechanical cycling power to be determined as a (discretized) time-continuous cycling power and/or a peak cycling power for each revolution.

At 320, at least one revolution can be identified. The at least one revolution can be identified as being associated with the plurality of force sensors.

The sensor readings may be acquired over the course of a plurality of revolutions for a given foot of a user while cycling. The sensor readings can be used to determine various data associated with individual revolutions.

The plurality of revolutions can be identified using data from the sensor readings. Each revolution may be identified based on accelerometer data included in the IMU sensor data acquired at 310. Each revolution may be identified as a time period between local signal minimums in the accelerometer data.

At 330, force values can be determined for the at least one revolution. The force values can be determined using aggregate force data based on the sensor readings.

For example, a force value may be determined for each revolution based on aggregate force data for the corresponding revolution. The sensor readings may include individual force sensor values from each of the force sensors in the plurality of force sensors. The aggregate force data may be determined based on the individual force sensor values received from the plurality of force sensors over the course of the revolution. For example, the aggregate force data for a given revolution may be determined as a sum of the individual force sensor values received from each force sensor.

The sensor readings may include a plurality of individual force sensor values from each of the force sensors at various time points throughout the revolution (e.g. as time-continuous sensor readings or sensor readings at discrete time steps). This can enable a plurality of force values to be determined for each revolution. Each force value may correspond to a particular time step (e.g. discrete or continuous point in time) in the revolution. The force value for a particular time step can be determined using the individual force sensor values received for that particular time step. For instance, each force value can be determined as a sum of the individual force sensor values received from each force sensor at the corresponding time step. Alternatively, discrete force values such as a peak force value or a mean force value may be determined.

At 340, cycling cadence, foot velocity and foot angle can be determined. The cycling cadence, foot velocity and foot angle can be determined separately for each revolution identified at 320. In some cases, each of the cadence, foot velocity and foot angle can be determined using the sensor readings acquired at 310.

The cadence generally represents the speed of rotation of the pedal for a given revolution. The cadence can be determined based on the corresponding number of revolutions that would result from the speed of pedal rotation in a given time period (e.g. revolutions per minute).

The cadence value may be determined in various ways. For example, where a user is cycling on a stationary bicycle, the cadence value may be determined as the cadence measured by the stationary bicycle for the given revolution. In some cases, the cadence value for a real bicycle may be determined using a cadence monitoring system coupled to the pedals or crank of the bicycle.

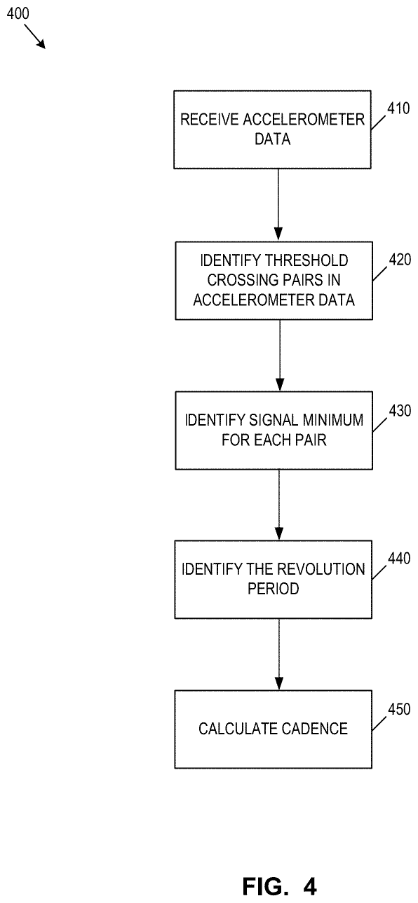

Alternatively, the cadence value may be determined based on sensor data received at 310. For each revolution, the cadence value may be determined by identifying a revolution period of the corresponding revolution. The revolution period may be determined using the sensor data acquired at 310. An example process 400 for determining the cadence using sensor data is described in further detail herein below with reference to FIG. 4.

The foot angle value can be determined using sensor data received at 310. The sensor data can include gyroscope data received from at least one IMU. A foot angle can be determined by integrating the gyroscope data in the sagittal plane (i.e. a plane defined by the forward-rearward direction and the upward-downward direction). The sagittal plane may be determined as the x-coordinate of the gyroscope from which the gyroscope data is received.

The foot angle may be determined as an estimated foot angle using an unknown integration constant. Alternatively, the integration constant may be calculated to allow the foot angle to be determined. The integration constant may be calculated using an average foot angle for a given revolution. The average foot angle may be estimated to be zero degrees for each revolution.

The sensor readings may include a plurality of gyroscope sensor values corresponding to various time points throughout the revolution (e.g. as time-continuous sensor readings or sensor readings at discrete time steps). Accordingly, a plurality of foot angle values may be determined for each revolution. Each foot angle value may correspond to a particular time step (e.g. discrete or continuous point in time) in the revolution. Each foot angle value may be determined by integrating the gyroscope data for the corresponding time step.

The foot velocity values can be determined using sensor data received at 310. The sensor data can include accelerometer data received from at least one IMU. The foot velocity values can be determined by integrating the accelerometer data over the period of the revolution.

The foot velocity values can include a plurality of direction-specific velocity values. Each direction-specific velocity value can be provided as a separate input to the machine learning model. The direction-specific velocity values can include a first foot velocity value in a forward-rearward direction and a second foot velocity value in an upward-downward direction. The first foot velocity value and the second foot velocity value can be provided as separate inputs to the machine learning model.

The direction-specific velocity values may be calculated solely for directions along the sagittal plane (e.g. based on accelerometer signals in the y- and z-directions). Lateral velocity (and accelerometer signals in the x-direction) can be omitted, as foot motion relating to the cycling power will generally only occur in the sagittal plane while cycling.

The first foot velocity value can be determined by integrating the accelerometer data in the forward-rearward direction. The first foot velocity value may be an estimated velocity value (e.g. because the integration constant is unknown). Alternatively, the integration constant may be calculated to allow the first foot velocity value to be determined. The integration constant may be calculated using a first average velocity value in the forward-rearward direction for a given revolution. The first average velocity value may be estimated to be zero for each revolution.

The second foot velocity value can be determined by integrating the accelerometer data in the upward-downward direction. The second foot velocity value may be an estimated velocity value (e.g. because the integration constant is unknown). Alternatively, the integration constant may be calculated to allow the second foot velocity value to be determined. The integration constant may be calculated using a second average velocity value in the upward-downward direction for a given revolution. The second average velocity value may be estimated to be zero for each revolution (each average directional component of the velocity may be estimated to be zero for each revolution).

The sensor readings may include a plurality of accelerometer values corresponding to various time points throughout the revolution (e.g. as time-continuous sensor readings or sensor readings at discrete time steps). Accordingly, a plurality of first foot velocities and a plurality of second foot velocities may be determined for each revolution. Each first foot velocity value and each second foot velocity value may correspond to a particular time step (e.g. discrete or continuous point in time) in the revolution. Each foot velocity value may be determined by integrating the accelerometer data for the corresponding time step.

The foot velocity values determined at 340 can be defined in the local coordinate system of the inertial measurement unit. Because cycling power relates to the power applied to the pedals of the bicycle which move along a consistent sagittal plane in the local coordinate system, there may be no need to convert the IMU data to a global coordinate system or reference frame.

Alternatively, the velocity values determined at 340 can be defined in a global reference frame. Accordingly, the accelerometer data can be transformed to a global reference frame prior to integration. The foot velocity values can be determined by transforming the accelerometer data to a global reference frame in order to generate transformed accelerometer data, then by integrating the transformed accelerometer data over the revolution period.

The accelerometer data may be transformed to the global reference frame based on the angle of the user's foot when the accelerometer data is acquired. The foot angle can be determined using gyroscope data received from the IMU. The accelerometer data may be transformed to the global reference frame using only the x-directional component of the gyroscope data (i.e. the component of the gyroscope data corresponding to the sagittal plane). For example, the global acceleration components a_{X},a_{Y},a_{Z} can be determined from the local components aₓ,a_{y},a_{z} of the accelerometer data according to:

$$[ \begin{matrix}{a}_{X} \\ {a}_{Y} \\ {a}_{Z}\end{matrix} ] = [ \begin{matrix}1 & 0 & 0 \\ 0 & cos ( {θ}_{x} ) & - s in ( {θ}_{x} ) \\ 0 & sin ( {θ}_{x} ) & cos ( {θ}_{x} )\end{matrix} ] \times [ \begin{matrix}{a}_{x} \\ {a}_{y} \\ {a}_{z}\end{matrix} ] - [ \begin{matrix}0 \\ 0 \\ g\end{matrix} ]$$

The first foot velocity value v_{Y} can then be determined according to:

v_{Y}=∫a_{Y}dt+C₂

where the integration constant C₂ may be left as an unknown or alternatively calculated, as explained herein above. The integration can be performed over the corresponding revolution period.

The second foot velocity value v_{Z} can be determined according to:

v_{z}=∫a_{Z}dt+C₃

where the integration constant C₃ may be left as an unknown or alternatively calculated, as explained herein above. The integration can be performed over the corresponding revolution period.

At 350, the force values (from 330) and the cycling cadence, foot velocity and foot angle (from 340) can be input to a machine learning model along with the user mass. The machine learning model can be trained to predict the mechanical cycling power in response to receiving the mass, force values, cycling cadence, foot velocity and foot angle as inputs. Accordingly, the mechanical cycling power associated with the plurality of sensor readings can be determined by the machine learning model.

The mechanical cycling power may be defined in various ways. For example, the mechanical cycling power may be defined as a peak mechanical cycling power for each revolution. The machine learning model can be trained to output the peak cycling power for each revolution based on the force values, the user mass, the cycling cadence, the foot velocity and the foot angle value determined for the entire revolution. Determining the mechanical cycling power as a peak mechanical cycling power can provide a user with useful biometric feedback, particularly since peak mechanical cycling power is related to the user's oxygen intake.

Alternatively or in addition, the mechanical cycling power can be determined as a time-continuous mechanical cycling power for each revolution. In such cases, the inputs to the machine learning model can also be determined on a time-continuous basis. The time-continuous mechanical cycling power may be defined using a fully continuous time scale or a discretized time scale that includes time steps. This may provide more granular data for feedback and analysis (e.g. enabling a cyclist to understand where in a crank cycle they generate the most power) with the trade-off of requiring increased computational expense.

Various different types of machine learning models may be used to determine the mechanical cycling power. For example, a non-linear machine learning model such as a neural network model may be used. Alternatively, a different type of machine learning model could be used to determine the mechanical cycling power, such as, for example a support vector machine, a gradient boosted decision tree, and so on.

The machine learning model can be trained used training data that includes the set of inputs (e.g. the force values, cadence, foot velocity values, foot angle values) determined from sensor data along with the mass of the user generating the corresponding training data. The inputs can be determined based on training sensor readings from a plurality of force sensors positioned underfoot and an IMU (as at 310) and preprocessing of the training sensor readings as described at steps 330 and 340 above (and method 400 below).

The training data can also include measured data representing the force output by a user. A training mechanical cycling power can be determined using pedals equipped with biometric sensors. For example, the training mechanical cycling power may be measured using instrumented force pedals.

The training data can be collected from one or more users performing an activity. Once the machine learning model is trained using the training data, the machine learning model can be applied to determine the mechanical cycling power of the same or different users performing an activity.

The training process may vary depending on the type of machine learning model being implemented. For example, with a neural network model, an optimization algorithm can be applied to optimize the connection weights between the neurons and between layers of the neural network model. The optimization algorithm may employ a cost function based on the difference between the desired outputs (as calculated from the pedal sensors data) and the model outputs (as calculated from the given inputs). The trained model can then be used, as at 350, to determine the mechanical cycling power based on preprocessed data from the plurality of force sensors (and the IMU).

Different combinations of input data may be used to train and implement the machine learning model. For example, the machine learning model may be trained to receive 6 inputs, namely a user mass value, a force value, a cadence value, a foot angle value, and two foot velocity values (e.g. one for each direction along the sagittal plane).

In some cases, the inputs to the machine learning model can include revolution-specific values for each preprocessed input. Thus, the preprocessing described in steps 330 and 340 can be performed for each revolution for which a mechanical cycling power is determined. Inputs such as the user mass may remain constant for all revolutions for a given user (or at least for a given user within a given session of a cycling activity).

In some cases, the preprocessed inputs may be determined as a single value for each revolution. For instance, a single cadence value can be determined for a given revolution. The cadence value may be determined as an average cadence for that revolution.

In some cases, the preprocessed inputs may include multiple values for each revolution. For instance, preprocessed inputs may be determined as time-continuous inputs that include multiple values at each time point or time step in a given revolution. For instance, foot angle values, foot velocity values, and force values may be determined as time-continuous inputs.

Alternatively, a different number of inputs may be used. For example, a different number of inputs can be used where additional inputs (e.g. acceleration, crank arm length) are included in addition to the example inputs described herein above.

Alternatively or in addition, some of the inputs may be adjusted or modified. For example, peak or mean force values may be used as an input in place of time-continuous force values.

For example, an estimated mechanical cycling power may be calculated directly based on the sensor readings from the force sensors and an IMU. The estimated mechanical cycling power may be calculated in various ways, e.g. as a peak power for a given revolution or a time-continuous power. Typically, the estimated mechanical cycling power to be calculated is defined in the same manner as the mechanical cycling power being output by the machine learning model. The estimated mechanical cycling power can be provided to the machine learning model as an additional input (e.g. along with the foot angle values, foot velocity values, and force values).

The estimated mechanical cycling power can be calculated based on the force values and components of the foot velocity. For instance, the first foot velocity value and the second foot velocity value can be used to calculate the estimated mechanical cycling power. As explained above, the first foot velocity value and the second foot velocity value can be determined for each revolution based on the inertial measurement data.

The estimated mechanical cycling power can be calculated using directional force components determined from the force values. To calculate the directional force components, unit vectors are determined corresponding to the forward-rearward direction (u_{Y}) and to the upward-downward direction (u_{Z}). The unit vectors can be determined to represent the portion of the total force that is applied in the corresponding direction at a given time.

The unit vectors are determined on the basis that the sensor forces act perpendicular to the sensor unit. Accordingly the unit vectors are determined by adding 90 degrees to the sagittal plane angle (θₓ). The unit vectors can be determined according to:

u_{Y}=cos(θₓ+90°)

u_{Z}=sin(θₓ+90°)

To determine the respective directional components of the force, each unit vector can be multiplied by the total force as determined from the sensor readings. A first directional force component F_{Y} in the forward-rearward direction can be determined from the magnitude of the total force values (F_{mag}) according to:

F_{Y}=−F_{mag}×u_{Y}

A second directional force component F_{Z} in the upward-downward direction can be determined from the magnitude of the total force values according to:

F_{Z}=−F_{mag}×u_{Z}

The magnitude of the total force values (F_{mag}) represents the reaction force measured by the sensors. However, the corresponding action force is used to calculate the cycling power. Accordingly, the negative value of the magnitude of the total force values is used to calculate the directional force values.

For example, the estimated mechanical cycling power P can be calculated according to:

P=F_{Y}×v_{Y}+F_{Z}×v_{Z}

where v_{Y} represents the first foot velocity value, v_{Z} represents the second foot velocity value, F_{Y} represents a first directional force component in the forward-rearward direction determined from the force values, and F_{Z} represents a second directional force component in the upward-downward direction determined from the force values.

Alternatively, the estimated mechanical cycling power can be calculated based on a torque value determined from the IMU data and the force values. The torque value (τ_{C}) can be multiplied by the average angular velocity (ω_{avg}) in order to provide the estimated mechanical cycling power according to:

P=τ_{C}×ω_{avg}

The torque value (τ_{C}) can be determined as a cross product between the time-continuous position of the pedals (d_{Y}, d_{Z}) and time continuous directional force components (F_{Y}, F_{Z}). For instance, the torque value (τ_{C}) can be determined according to:

τ=F_{Z}×d_{Y}−F_{Y}×d_{Z}

The time-continuous position of the pedals can be determined using the time-continuous velocity of the pedals. The time-continuous velocity of the pedals can be determined as the first foot velocity value and the second foot velocity value. The time-continuous position of the pedals can then be determined by integrating the first foot velocity value and the second foot velocity value according to:

d_{Y}=∫v_{Y}dt+C₄

d_{Z}=∫v_{Z}dt+C₅

At 360, the mechanical cycling power determined by the machine learning model at 350 can be output. This may provide a user with data representing their power output when performing a cycling activity. The output data can provide the user with insight into their level of performance while cycling. The output data can additionally be used as an input to a game.

The mechanical cycling power can be output directly through an output device to provide a user with feedback on the activity being monitored. For example, the mechanical cycling power may be transmitted to a mobile application on the user's mobile device (e.g. a processing device 108), allowing the user to continuously monitor and improve while performing the activity. Providing a user with real-time feedback, or near-real-time feedback may assist the user in maintaining a high-level of performance for an extended period of time. For example, providing a user with such feedback can allow the user to perform at their maximum capacity without rapidly fatiguing or burning out by maintaining a consistent cycling power across varied terrain. Mechanical cycling power can also be used for zone training. Zone training is a type of athletic training which encourages users to keep their metrics within a range or “zone” of values over a predetermined period of time (e.g. the length of a game). Users may be shown their position in a zone in real-time (e.g. on a processing device 108) and may be rewarded for staying within the zone and/or penalized for leaving the zone. For example, a user may be given a mechanical cycling power zone to stay within for a cycling game. During the game, the user will be encouraged to keep their mechanical cycling power in the designated zone to achieve maximum points. Alternatively or in addition, the mechanical cycling power may be stored, e.g. for later review, comparison, analysis, or monitoring.

Optionally, additional derived metrics can also be output. In some cases, time-continuous data reflecting where in a given revolution power or torque is applied may also be determined. For example, the torque applied at various points during a revolution may be determined and provided to a user as feedback.

Where torque is applied during a revolution can be determined based on a time-continuous crank angle. The time-continuous crank angle may be determined based on the time-continuous position of the pedals. The time-continuous crank angle (θ_{c}) may be determined by applying an arc-tangent to the time-continuous position of the pedals according to:

θ_{c}=atan 2(d_{Z},d_{Y})+180 °

where 180° are added to the crank angle so that top dead-centre occurs at 90° rather than 270°. The crank angle can then be compared against the torque, e.g. in an output plot in a cartesian or polar coordinate system.

The example method 300 described herein above generally describes the process of determining mechanical cycling power for one leg. Optionally, method 300 may be applied to determine the mechanical cycling power for both of a user's legs based, respectively, on data (e.g. sensor readings and IMU data) collected for each leg. Method 300 may be performed concurrently on the data collected for each leg in order to provide a user with real-time feedback of the mechanical cycling power generated by each leg.

Referring now to FIG. 4, shown therein is an example method 400 of determining cadence for a user performing a cycling activity. Method 400 may be used with a method of determining mechanical cycling power such as method 300 for example. The method 400 may be applied to determine the cadence for an individual revolution.

The example method 400 is an example of a method for determining cadence using inertial measurement data received from an IMU arranged to measure the position and/or motion of a user's foot. The inertial measurement unit can include at least one accelerometer. The inertial measurement unit may be associated with a plurality of force sensors positioned underneath the user's foot.

At 410, accelerometer data can be received from at least one inertial measurement unit. The length of a given revolution (also referred to as the revolution period) can be identified using the z-direction component (the component in the upward-downward direction) of the accelerometer signal received from the at least one inertial measurement unit.

At 420, a plurality of threshold crossing pairs can be identified in the accelerometer data received at 410. Each threshold crossing pair can include a first threshold crossing point and a second threshold crossing point in the accelerometer data. Each threshold crossing point can be identified as a point in the accelerometer data where the accelerometer signal crosses a specified threshold (also referred to as a cadence threshold).

The first threshold crossing point can be identified as a first point (e.g. a point in time or time step) when the accelerometer data crosses the specified cadence threshold while the accelerometer signal is decreasing. The second threshold crossing point can be identified as a second point (e.g. a point in time or time step) when the accelerometer data crosses the specified cadence threshold while the accelerometer signal is increasing.

The specified cadence threshold may be defined as a specified acceleration value. That is, when the acceleration signal indicates that acceleration is changing and crosses the specified acceleration value, a threshold crossing point can be identified. The specified acceleration value can be defined as an absolute acceleration value.

In some examples, the specified acceleration value may be an absolute acceleration signal value defined to confirm that cycling is occurring. For example, the specified acceleration value may be defined as about 0.8 g. In some cases, slightly different values of the specified acceleration value may be selected while still ensuring that cycling is occurring.

If the received acceleration signal indicates a value of about 1 g over an extended period of time, this indicates that the IMU is at rest (i.e. the user has stopped cycling). However, if the acceleration signal continues to vary about 1 g (e.g. continually exceeds 1 g and then drops below 1 g) this can indicate that the user is cycling. Selecting a specified acceleration value slightly below 1 g (such as 0.8 g for example) may allow for some drift error in the acceleration signal (e.g. resulting in the resting acceleration not being exactly 1 g) while ensuring that the threshold remains indicative of cycling occurring.

At 430, a signal minimum can be identified for each threshold crossing pair identified at 420. The signal minimum can be identified from the accelerometer data received between the time of the first threshold crossing point and the time of the second threshold crossing point. The signal minimum can be identified at a signal minimum point located between the first point and the second point.

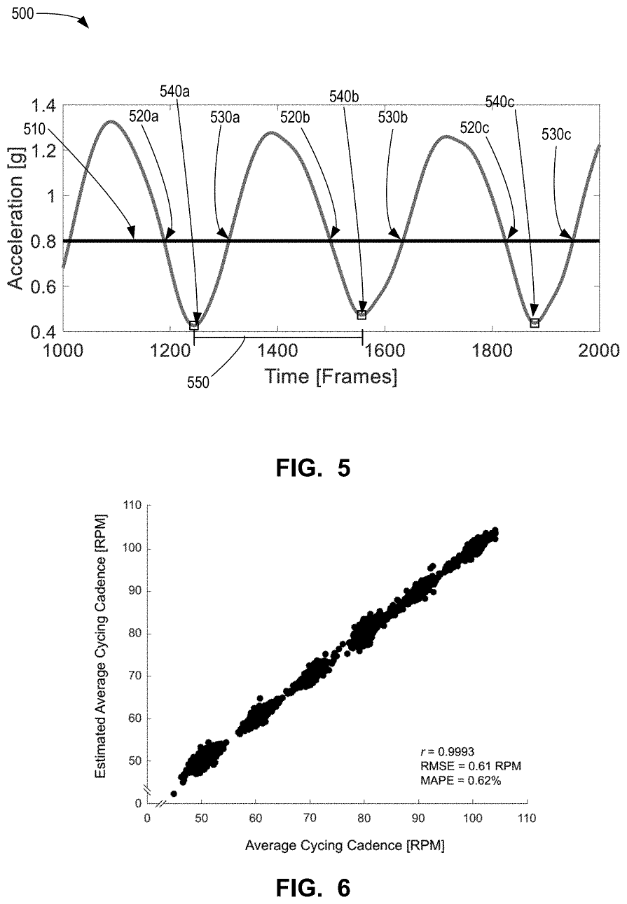

FIG. 5 illustrates an example plot 500 of the z-directional component of an acceleration signal received from an IMU, such as the signal received at 410. As shown in plot 500, a specified cadence threshold 510 has been defined at about 0.8 g. A plurality of threshold crossing pairs (520a and 530a , 520b and 530b , and 520c and 530c ) have been identified using the specified cadence threshold 510. Each threshold crossing pair includes a first threshold crossing point 520 and a second threshold crossing point 530.

Signal minimums 540a -540c have also been identified for each threshold crossing pair. As shown in the example of plot 500, the signal minimum 540 can be identified as the local minimum (e.g. the lowest value point) in the acceleration signal at a time between the first threshold crossing point 520 and second threshold crossing point 530 of the given threshold crossing pair.

Referring again to FIG. 4, at 440 a revolution period of the corresponding revolution can be identified. The revolution period can be identified as the period extending between the signal minimums of adjacent threshold crossing pairs.

The revolution period can be determined as the length of time (e.g. the time difference) between the times corresponding to the signal minimums. In the example of FIG. 5, a revolution period 550 is shown between the times corresponding to signal minimums 540a and 540b.

At 450, the cadence can be determined for the revolution. The cadence can be determined by converting the revolution period (from 440) to a cadence value. The cadence may then be provided as an input to the machine learning model at 350 to determine the mechanical cycling power.

The cadence can be determined as an average cadence over the course of a single revolution of a pedal, e.g. by relying on the length of the entire revolution period. For example, the length of the revolution period can be converted to the corresponding number of revolutions per minute to provide the cadence value.

The cadence may be determined individually for each foot. Alternatively, a cadence value for one revolution of the right foot and one revolution of the left foot can be averaged to define the cadence for the given revolution.

Optionally, the cadence can be output directly through an output device to provide a user with feedback on the activity being monitored. For example, the cadence may be transmitted to a mobile application on the user's mobile device (e.g. a processing device 108) allowing the user to continuously monitor and improve while performing the activity. Like mechanical cycling power, cadence can be used for zone training. A user may select or be assigned a cadence zone to maintain during an activity, such as a cycling game. During the game, the user will be encouraged to keep their cadence in the designated zone to achieve maximum points. Alternatively or in addition, the cadence may be stored, e.g. for later review, comparison, analysis, or monitoring.

The mechanical cycling power, cadence, additional derived metrics, or some combination thereof may be outputs (i.e. an output dataset) of the system.

The outputs can be used as an input to a game. A game could be a cycling training game, where a user is incentivized to improve their cycling technique and achieve new goals. Alternatively, a game could be a non-cycling focused game that requires a user to cycle at certain points in a game (e.g. a robbery game where a user must escape on a bicycle during one level). Methods 300 and 400 can be used to calculate mechanical cycling power and cadence for users riding both outdoor bicycles and indoor (stationary) bicycles while wearing the insoles, hence either type of bicycle can be used for gaming.

The way a user cycles in real life may be recreated in a game, based on their outputs. For example, an avatar in a game may cycle at the same cadence that a user cycles in real life. The user may need to adjust their cadence in real life in order to dodge obstacles or cycle along certain sections of a track in a game.

Virtual environments, objects, and avatars may be generated, with which a user using the system can interact. The virtual environment or surroundings and virtual objects can be altered based on the movements, gestures, and the outputs of the user. Output devices (e.g. a television screen, a virtual reality headset, etc.) may be used to display the virtual environment to users. A user may visit a variety of virtual environments, including imaginary environments or environments that replicate real-life environments (e.g. the Tour de France route). When a user moves around while wearing the carrier unit, they will move around in and interact with the virtual environment accordingly.

A scaling factor may be applied to outputs in a game. The scaling factor may be an integer (e.g. 1, 2, 5, 10, etc.) or it may not be an integer (e.g. 0.2, 1.5, 2.6, 6.9, etc.). In one example, the scaling factor may be 1. In this case, the outputs are applied equivalently in a game (i.e. a 1:1 scaling). The cycling cadence of an avatar in the game is equivalent to the cycling cadence of a user in real life. In another example, the scaling factor may be 5. In this case, outputs are scaled 1:5 from real life to the game. The cycling cadence of an avatar in the game is five times faster than the cycling cadence of a user in real life. Gaming experiences that are directly based on a user's outputs allow users to have a more realistic and immersive gaming experience than games that are not based on a user's biometric data (e.g. games played with buttons on a controller). Output scaling may allow for superhuman performance enhancements in a game. For example, an avatar whose cycling cadence is scaled by a scaling factor of 10 may be able to cycle faster than a car in a game, but an avatar whose cycling cadence is scaled by a scaling factor of 1 may not be able to. Different scaling factors may also be applied to different outputs. For example, a scaling factor of 2 may be applied to the cycling cadence, but a scaling factor of 0.5 may be applied to mechanical cycling power. (An avatar may be able to cycle faster and longer without burning out than a human, if the mechanical cycling power is scaled).

The outputs may also be applied to different environmental factors in a game. For example, the wind speed in a game can be changed. The outputs can be applied to the new environmental factors, so a user can understand how they might perform in a different environment. The performance of the user under the original conditions and the simulated conditions can be shown on a visual display.

The virtual environment can display or generate an avatar representing the portion of a user's body to which the carrier unit is affixed. For example, if the carrier unit is a pair of insoles, a user's feet may be rendered in the game environment. Features of a bicycle, such as pedals, may also be rendered in the game environment. A user may be able to change the skins and/or shoes applied to their feet in the game environment, or the look and features of their virtual bicycle. For example, a user may choose a special type of cycling shoe to be shown in the virtual environment. Special objects and/or abilities may be associated with the virtual skins, shoes, and bicycles. For example, virtual turbo engines may extend from a virtual bicycle for quickly fleeing from danger in a game. As another example, a virtual bicycle may contain a special feature, where it can build up energy if a user reaches certain cycling goals. The built-up energy can be expended as a burst of power for a user's avatar, when the user wishes to expend it in a cycling game.

Alternatively, the game environment can display or generate an avatar for the user's entire body. The appearance of the avatar's body may depend on the user's outputs. For example, if fast cadences are frequently recorded for a user, their avatar may appear aerodynamic (e.g. wearing tight clothing). An avatar's appearance may also be location dependent. For example, if a user lives in a warm, dry climate, the avatar may be depicted in shorts and a t-shirt, with dried sand on their skin. Alternatively, if a user lives in the Arctic, their avatar may be depicted in a parka and furry boots. There may be location-dependent virtual items that can be unlocked. For example, if a user travels to another country in real life, they may unlock a special virtual bicycle from that country. The carrier unit may contain a GPS system or another location-sensing system to enable the location-dependent items and features to be unlocked.

The outputs may also be used to model the dynamics of virtual objects and/or surroundings within a game, with which the user interacts. For example, if a user increases their cadence, their virtual surroundings in a game will appear to move faster by them. If a user's avatar cyclist collides with a pylon while cycling, the pylon will be thrown in a trajectory defined by the user's cycling outputs.

Additionally, the outputs may be used to control a character in a lifestyle game. These games may require a user to virtually embody a certain lifestyle and complete tasks involved with the lifestyle. For example, a user may embody the lifestyle of an Olympic cyclist in a game. The user will be required to train like an athlete, and the outputs can be used to determine if the user has successfully completed the training. They may also be required to complete other tasks relating to the lifestyle of an Olympic athlete, such as taking rest days, taking part in competitions, achieving sponsorships, going on press tours, going grocery shopping, etc.

The system may also contain safety features to prevent collisions while cycling or gaming, using sensors and/or software included in the carrier unit. In one embodiment, wherein the carrier unit is an insole, software for the Bluetooth system may detect if a user's pair of insoles is in close proximity to another user's pair of insoles. Alternatively, GPS systems embedded in the insoles and optionally other objects may detect if a user is at risk of colliding with another user or object. The system may alert users if they are at risk of a collision. For example, if two cycling users approach the same street corner from different directions, they may be alerted to a possible collision. In another example, if a cycling user approaches an intersection at the same time as a car (a user in the car may be wearing a pair of the sensorized insoles or the car's GPS system may communicate with the cycling user's insoles), the cycling user and/or the driving user may be alerted of a potential collision.

The system may be paired with other carrier devices in gaming scenarios. For example, the insoles may be paired with other wearable devices, such as wrist-worn IMUs. A gaming platform comprising multiple wearable game controllers at different locations on the body can encourage users to engage with a game using their full body, which may increase their workout and fitness during the game. The system may also be paired with fitness equipment. For example, the insoles can be paired with a stationary bicycle for a cycling game. The resistance of the stationary bicycle can change in response to different virtual terrains (e.g. cycling up a virtual mountain), and the user's outputs, as determined from the insoles, can determine how they are performing in the game. Visual display carrier units, such as VR headsets, smart glasses, and smart goggles, may also be paired with the insoles to increase the immersivity of games.

The system may also contain additional sensor types, whose data can be used to augment gaming experiences. In particular, temperature sensors may provide various advantages for health and gaming applications. The system may include one or more temperature sensors used to measure body or environmental temperature. In a first example, one or more temperature sensors (e.g. thermistors) may be included in a flexible printed circuit within the bulk of the insole. The one or more temperature sensors can detect temperature changes from the body. The temperature changes may be used in an algorithm that adjusts other sensor (e.g. force sensor) readings to account for temperature drift. Alternatively, the one or more temperature sensors may be used to measure the body temperature of users for health and gaming calculations (e.g. calorie burn calculations or task readiness calculations). In another example, the one or more temperature sensors may be affixed to the outside of the shoe or at other locations away from a user's body to determine the external temperature. The external temperature may be used in gaming to send safety messages and notifications to users (e.g. if the external temperature is hot, a user may receive a notification suggesting they hydrate more frequently). The external temperature may also be used to adjust health and gaming calculations and may be used to adjust the virtual environment in a game (e.g. if the external temperature is hot, the game may place the user in a virtual desert).

Outputs may contribute to scoring in a game. For example, a performance score may be calculated from the outputs. Improvements in these metrics may result in an increase in the number of points earned in a game, incentivizing users to improve and safely push their limits when cycling.

The performance scores can be accumulated based on a user's participation in various activities providing a standardized way to incentivize or reward users for movement in non-gaming (e.g. athletic) and gaming activities.

One or more normalization factors may be defined to allow the outputs and performance scores to be determined fairly for different users. Normalization factors may be applied to account for factors such as mass, weight, age, gender, natural athletic ability, game skill, other physical characteristics, or combinations thereof.

The calculation of performance scores can also include modification factors such as multipliers and bonuses for successful completion of objectives including streaks, skillful movement combinations, and/or other unique game experiences such that performing the same in-game action may not yield the same performance scores each time. For example, if a user completes a Tabata cycling sequence in a game according to the instructions, they may receive a 5× multiplier for all performance score points collected up to a minute after the sequence is completed.

The performance score and the outputs can also be used to determine other gaming-related metrics for a user. For example, a user can be associated with one or more user levels. The user levels generally refer to the experience of a user within a game. User levels may be used to compare users to one another, or to establish progression in fitness and experience over time.

The performance score and the outputs may also be used to generate and to track progress towards achieving training goals within a predetermined time period. For example, based on a user's mechanical cycling power values over one week, a training goal can be generated for the user to achieve the same or greater mechanical cycling power values the subsequent week. The user's mechanical cycling power can then be tracked the subsequent week to determine the user's percentage of progress towards achieving the training goal.

Training goals can relate to accumulated performance scores, outputs, system usage metrics, or any combination thereof that should be achieved in a predetermined time period (session, day, week, month, year, season, etc.) or instantaneous values (i.e. a rate) that should be achieved at a certain point in time. Training goals may be suggested by the processing system based on previous activities, be chosen by the user, or be presented as part of a challenge from another user or group of users. Suggested training goals can become increasingly targeted for users as additional sensor data is collected by the system over time.

Training goals can be directed toward weight loss. Wherein the carrier unit is an insole containing force sensors, body weight or mass can be measured by the insoles. Alternatively, an external device may be used to measure body weight or mass and transmit the values to the input device 102, remote processing device 108, or cloud server 110. If a user has a training goal to lose a certain amount of weight, the processing system may recommend certain activities to help them accomplish their goal. In particular, the processing system may recommend fitness-related games that can be played with the carrier unit. For example, for an overweight user, the system may suggest lower intensity cycling games. The system may create a fitness-based game schedule for the user to follow, to encourage increased activity and intensity as the user's body weight or mass decreases (i.e. as their percentage of progress towards achieving the training goal increases). The system may also include a virtual coach to help the user in their weight loss journey. A user may participate in virtual weight loss groups and/or rooms to encourage participation and support through interacting with other users with similar training goals. Weight loss may also be encouraged through badges, virtual gifts, streaks, and other virtual achievements.

Training goals may also be directed toward education. Specific games and activities may integrate educational concepts (e.g. a cycling game that helps users learn a new language). The same social interactions and virtual achievements in the weight loss example may also apply to a user's journey with an educational goal.

Additionally, the outputs may also be used to assess a user's technique when performing an activity or movement (i.e. their quality of movement). Wherein the carrier unit is an insole containing force sensors, a user's outputs may be recorded and stored in the system memory for an activity, such as cycling. As further data is collected for the user, the system may compare previous data against new data to determine differences in technique to notify the user of fatigue or of a potential injury. Alternatively, the system may compare data contralaterally (i.e. between opposing limbs) to determine differences in technique. To assess technique, a machine learning model may be trained on data that includes both “correct” and “incorrect” versions of an activity. In implementation, the model can then classify an activity as “correctly” or “incorrectly” performed. Alternatively, the model can be trained on data that includes rankings (e.g. by a clinician or sports scientist) on technique of certain activities (e.g. a 0 to 5 ranking, where 0 indicates that an activity was poorly executed and where 5 indicates that an activity was perfectly executed). In implementation, the system can reject exercise tasks below a certain ranking and/or output the ranked value. In another example, technique can be assessed based on conditions or restrictions set for each activity (e.g. timing of gear shifts, in order to maintain a certain mechanical cycling power during an uphill cycle). A user's outputs can be used to determine if the condition was met. If the user does not meet the condition or restriction, their technique may be deemed unacceptable.

In a further example, the outputs may also be used to determine a user's “readiness” to participate in a game or activity. For example, a user's historical mechanical cycling power data for 20 km bike rides may be used to determine a user's readiness to cycle a 50 km bike ride. Alternatively, other data that can be collected from the insoles (e.g. center of pressure, ground reaction force, etc.) can be used to determine a person's readiness to participate in a cycling game.

Task readiness refers to a user's ability to perform a task at a moment in time. It is assessed by having a user perform a task, such as a jump, squat, balance, sprint, series of cycling revolutions, or another physical exercise with the carrier device. The exercise may be included as part of a game or challenge or may be separate from game play. Injury potential, technique, and/or fatigue state of the user may be incorporated in a task readiness score or may be pulled out of the task readiness score and displayed as a separate score. The task readiness, injury potential, technique, and/or fatigue state scores may be recorded over time and may be displayed in a metrics report. The metrics report may be used to quantify improvements and overall fitness. The real-time readiness scores of the user may be reported to the user on the input device 102, remote processing device 108, or cloud server 110. For example, on a display of the remote processing device, a poor task readiness score may be reported as a red bar, an average task readiness score as a yellow bar, and a good task readiness score as a green bar in the top corner of the display. The task readiness feedback may alert the user to a deteriorating quality of their movements, which can be used to make an informed decision on continuation of game play. The task readiness scores may be used to recommend games that are appropriate for the user's physical state (e.g. their fitness level) at a certain point in time. For example, consistently high task readiness scores over a period may indicate that a user should play more advanced games to improve their fitness level. The system may recommend more advanced games to the user or higher-level players to compete against. The task readiness scores may also be used to recommend rest periods for the user or to coach the user through auditory means, visual means, tactile means, or some combination thereof. For example, a virtual coach may use task readiness scores to instruct the user on how to improve movement quality to gain more points, prevent injury, or achieve another goal in the game.

A virtual coach may be used to assist a user with meeting their training goals. The virtual coach may be trained through machine learning or other algorithms to give suggestions, notifications, and encouragement to the user relating to the training goal. Alternatively, a personal trainer, physiotherapist or other expert in the field may assess a user's historical outputs to develop and suggest training goals and paths to achieving training goals within the game.

Feedback may also be provided to users based on their outputs. For example, a user may be assigned a goal of staying within a certain mechanical cycling power zone for a ride or a series of rides. If the user begins to fall outside of that zone, they may be alerted. Feedback may be provided in the form of haptic feedback, such as with vibrational motors embedded in the carrier unit.

Feedback may also be provided in the form of an audio signal. A user's outputs may be sonified and played in real-time or post-activity for the user. For example, if a user goes on a ride, their mechanical cycling power can be sonified and played in real time. The user can then sonically identify changes in their mechanical cycling power, and they can make real-time adjustments to their cycling technique to maintain or improve their performance. Signal processing techniques may be used to increase the effects of sonification. For example, signals may be amplified, such that the sonification spans a broader range of tones than an unamplified signal, which may make it easier for users to identify changes in tone. Signals may also be layered. For example, the signals from the right and left foot may be added together prior to sonification, or the sonifications from the right and left foot may be played simultaneously. Signals may also be filtered to minimize noise, which may be distracting to a user once the signal is sonified. Visual feedback may also be provided by the system.

Users may review their feedback and data (e.g visualizations, sonifications, and haptics) during or after an activity. Real-time feedback may encourage users to continue to engage with the activity at a higher level of intensity or to increase their intensity. Post-activity data reviews may encourage users to understand their activity and movement statistics to prepare for improvements in the next activity.

Sonification of the outputs may also be used for artistic purposes. For example, these metrics may correspond to certain musical features, such as notes, instruments, tempos, and volumes. In one embodiment, cadence may control the tempo of the music. As a user cycles faster, the tempo of the music will increase. Mechanical cycling power may control the volume of the music. As a user's mechanical cycling power increases, so will the volume of the music. Users may work together to create music. For example, if two users go cycling, one user's sonification may create a melody and the other user's sonification may create a harmony. In this regard, users can generate music in real time with their bodies. Similarly, users, such as DJs, may be able to mix music in real time. For example, a DJ may cycle on a stationary bicycle at a concert while wearing the insoles, and by changing their cycling technique, they can cue tracks and increase or decrease the speed of tracks.

The outputs may also be used to create visualizations. Visualizations may be data driven (e.g. graphs) or artistic visuals (e.g. mandalas inspired by the spinning bicycle wheels, whose designs change according to the outputs of a user). For example, a large display screen may be used to show a user's outputs while they are cycling, racing, or gaming.

Additionally, information may be communicated to and/or between users through visual, audio, or haptic cues. For example, the system may send a haptic cue to a user's insoles to prompt them to complete a daily challenge based on their outputs. The results of their daily challenges may be compared with the results of other users. Alternatively, if cues are sent between users, a first user in a game may challenge a second user in a game to perform an activity by sending a haptic signal to the second user's carrier device. The communicated information may be based upon the two users' outputs. For example, the first user may send a haptic cue to the second user to challenge them to a ride, where the user with the highest mechanical cycling power during the cycle will be declared the winner.

Users may also be able to create levels or challenges for other users based on their outputs. For example, a first user may create a challenge that involves cycling up a steep hill. If a second user wishes to perform the challenge, they will be required to produce outputs (such as mechanical cycling power) that indicate that they performed an equivalently challenging or more challenging task (e.g. cycling faster up a hill of the same incline).

The outputs and/or performance scores may be displayed on an output device, as part of the remote processing device 108 or cloud server 110. A user may also be able to interact with a visual display via an interactive medium (e.g. a touchscreen) on the output device. Examples of data visualizations that may be provided on the visual display based on the performance scores and/or outputs of a user using the carrier unit include: foot pressure maps to show the pressure distribution on the insoles, foot pressure maps to show the movement of the center of pressure, points displays (e.g. performance score), pop-up notifications of errors in movement, pop-up notifications with suggestions to correct the movement, graphs showing changes in data over time, colour codes (e.g. different colour pop-ups for different performance scores or gestures), footprints whose shapes or depths are estimated based on the force sensor readings and/or derived force values, cumulative displays (e.g. accumulation of mechanical cycling power, which, when a certain level is reached, may be used to provide a burst of power for an avatar in a game), or combinations thereof. The data visualizations may be altered or enabled or disabled by users, with toggles, buttons, or other actions.

The output device may also display information (such as names, outputs, etc.) of other users in the same area using the same type of system. Carrier units may contain GPS systems or other location-sensing systems to enable viewing information of other users in the same area. Location-sensing may provide opportunities for virtual social interactions between users. Examples of social interactions include gift exchanges, meet-ups in virtual rooms, messaging, game challenges, cooperative games, competitive games, combination games (i.e. games with a competitive and cooperative aspect), tournaments, leaderboards (e.g. for age groups, geographic locations, specific games, etc.), and the ability to “follow” and/or “friend” other users (i.e. adding users to a list of “friends” on the system platform). Other social interactions known in the art, but not listed here, may also be included.

Virtual meeting rooms are digital areas where users may send messages or chats with one another, play games together, and participate in social interactions with other users. The system may have virtual meeting rooms available, or users may create and design their own virtual meeting rooms. The owner of a virtual meeting room may allow open access to the virtual meeting room, or they may restrict access to certain users. The owner may invite users to join their virtual meeting room.

Social interactions may also include competitive races against the outputs of the same user (i.e. their previous scores), other users, a “computer”, celebrities, and/or professionals. For example, a user may enable a “ghost” mode, where they can view their previous performances when repeating an activity, to compete against themselves. In a game where a user is required to perform a cycling game, they can view a “ghost” of their avatar's best performance while repeating the activity, along with an overlay showing the ghost's outputs to encourage them to match or improve from the previous performance. In another example, in the cycling game, the user may enable “ghost” mode to view the outputs of a professional cyclist, who recorded their outputs in the game for other users to copy. The user can work towards matching the professional cyclist's data to improve their own performance. In another example, a professional cyclist may create a virtual competition where users can compete against the professional cyclistfor a month-long cycling challenge. The participating users' outputs can be compared to the professional's outputs to determine if any of the users beat the professional. Users who participate in and/or win the challenge may receive a virtual reward.

### EXAMPLES

An implementation of the systems and methods described herein were tested. In particular, an implementation of method 400 using an IMU mounted in an insole was tested to evaluate the accuracy of determining the cycling cadence as compared to the cadence measured by Sensix™ pedal sensors. The implementation of method 400 was also tested using the same cycling data collected from 10 users, who cycled at 6 different cadences (50, 60, 70, 80, 90, 100 RPM) and at 3 different powers (150, 200, 250 W).

FIG. 6 shows a comparison of the cadence as calculated with the example implementation of method 400 (y-axis) compared to the cadence measured by the Sensix™ pedal sensors (x-axis). As illustrated in FIG. 6, the cadence calculated with the example implementation of method 400 has a low level of error (a root-mean square error of 0.61 RPM and a mean absolute percentage error of 0.61%). There is also a high correlation (R=0.9993) between the cadences calculated by the two methods, further demonstrating that the implementation of method 400 is highly accurate.

An implementation of method 300 was also tested using a plurality of force sensors and an IMU mounted in an insole and a neural network model trained to generate a peak cycling power for each revolution using 7 inputs (a user mass, a cadence value, a time-continuous force value, a time-continuous foot angle, a time-continuous velocity value in the forward-rearward direction, a time-continuous velocity value in the upward-downward direction, and an estimated mechanical cycling power) to evaluate the accuracy of determining the mechanical cycling power.

The peak mechanical cycling power determined by the tested implementation of method 300 was compared against the peak mechanical cycling power measured by pedals equipped with Sensix™ pedal sensors. The implementation of method 300 and the SensixTM pedal sensors were both configured to determine the peak cycling power for each revolution. Cycling data was collected from 10 users who cycled at 6 different cadences (50, 60, 70, 80, 90, 100 RPM) and at 3 different powers (150, 200, 250 W).

FIG. 7 shows a comparison of the peak power as determined using the example implementation of method 300 (y-axis) compared to the peak power determined by the Sensix™ pedal sensors (x-axis). As shown in FIG. 7, the plot shows fairly linear results indicating reasonable accuracy in the estimated peak power as determined by the example implementation of method 300 (see also Table 1 below).

FIG. 8 shows a comparison of the peak power as determined using a physics-based approach (y-axis) compared to the peak power determined by the Sensix™ pedal sensors (x-axis). The physics-based power was computed as the multiplication of the forces and velocities in their respective directions according to:

P=F_{Y}−v_{Y}+F_{Z}×v_{Z}

As shown in FIG. 8, the estimated peak power determined using the physics-based approach alone is substantially less accurate as compared to the peak power determined by the example implementation of method 300 (as shown by FIG. 7).

Table 1 below illustrates the correlation, root-mean square error (RMSE) and mean absolute percentage error (MAPE) determined through testing of the example implementation of method 300 (shown in FIG. 7) and the physics-based approach (shown in FIG. 8).

TABLE 1
Correlation and Error Values of Example
Methods of Calculating Cycling Power

| | Correlation | RMSE [W] | MAPE [%] |
|---|---|---|---|
| Implementation of Method | 0.84 ± 0.06 | 60.59 ± 12.44 | 11.86 ± 2.59 |
| 300 | | | |
| Physics-Based Approach | −0.38 ± 0.11 | 280.37 ± 28.21 | 57.25 ± 6.51 |

As shown in Table 1, the example implementation of method 300 shows a substantial increase in correlation and a substantial reduction in error as compared to the physics-based approach.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A method for determining cycling power using force sensor data from a plurality of force sensors positioned underfoot, the method comprising:
identifying at least one revolution associated with the plurality of force sensors;
obtaining a plurality of force sensor readings from the plurality of force sensors during the at least one revolution;
determining force values for the at least one revolution using aggregate data based on the force sensor readings;
determining a user mass associated with the plurality of force sensor readings;
for each revolution, determining a cycling cadence, a foot velocity, and a foot angle; and
determining a mechanical cycling power associated with the plurality of force sensor readings by inputting the force values, the user mass, the cycling cadence, the foot velocity and the foot angle to a machine learning model trained to predict the mechanical cycling power.

2. The method of claim 1, further comprising outputting an output dataset, wherein the output dataset comprises the mechanical cycling power and/or the cycling cadence, and wherein the output dataset is used as an input to a game.

3. The method of claim 1, wherein the cycling cadence is determined by:
receiving accelerometer data from at least one inertial measurement unit associated with the plurality of force sensors;
identifying a revolution period of the corresponding revolution by:
identifying a plurality of threshold crossing pairs, each threshold crossing pair including a first threshold crossing point and a second threshold crossing point, wherein the first threshold crossing point is identified as a first point when the accelerometer data crosses a specified cadence threshold while decreasing, and wherein the second threshold crossing point is identified as a second point when the accelerometer data crosses the specified cadence threshold while increasing;
for each threshold crossing pair, identifying a signal minimum from the accelerometer data at a signal minimum point located between the first point and the second point; and
identifying the revolution period as a period extending between the signal minimums of adjacent threshold crossing pairs;
identifying the cycling cadence for the corresponding revolution by converting the revolution periods to a cadence value.

4. The method of claim 1, wherein the foot angle is determined by:
receiving gyroscope data from at least one inertial measurement unit associated with the plurality of force sensors; and
determining the foot angle by integrating the gyroscope data in a sagittal plane.

5. The method of claim 1, wherein the foot velocity comprises a first foot velocity value in a forward-rearward direction and a second foot velocity value in an upward-downward direction, and the first foot velocity value and the second foot velocity value are provided as separate inputs to the machine learning model.

6. The method of claim 5, wherein the first foot velocity value is determined by receiving accelerometer data from at least one inertial measurement unit associated with the plurality of force sensors and by integrating the accelerometer data in the forward-rearward direction; and wherein the second foot velocity value is determined by receiving accelerometer data from the at least one inertial measurement unit and by integrating the accelerometer data in the upward-downward direction.

7. The method of claim 5, further comprising:
calculating an estimated mechanical cycling power using the first foot velocity value, the second foot velocity value, and the force values; and
wherein determining the mechanical cycling power comprises inputting the estimated mechanical cycling power to the machine learning model along with the force values, the user mass, the cycling cadence, the foot velocity and the foot angle.

8. The method of claim 7, wherein the estimated mechanical cycling power P is calculated according to:
P=F_{Y}−v_{Y}+F_{Z}×v_{Z}
where v_{Y} represents the first foot velocity value, v_{Z} represents the second foot velocity value, F_{Y} represents a first directional force component in the forward-rearward direction determined from the force values, and F_{Z} represents a second directional force component in the upward-downward direction determined from the force values.

9. The method of claim 1, wherein the machine learning model is a neural network model.

10. The method of claim 1, wherein the machine learning model is trained using training data generated by collecting training sensor readings and a training mechanical cycling power calculated from pedals equipped with force sensors.

11. The method of claim 1, wherein the mechanical cycling power is determined as a peak mechanical cycling power or a time-continuous mechanical cycling power for each revolution.

12. A system for determining cycling power, the system comprising:
a plurality of force sensors positionable underfoot;
one or more processors communicatively coupled to the plurality of force sensors; and
a non-transitory storage memory storing a machine learning model trained to predict mechanical cycling power;
wherein the one or more processors are configured to:
identify at least one revolution associated with the plurality of force sensors;
obtain a plurality of force sensor readings from the plurality of force sensors during the at least one revolution;
determine force values for the at least one revolution using aggregate data based on the force sensor readings;
determine a user mass associated with the plurality of force sensor readings;
for each revolution, determine a cycling cadence, a foot velocity, and a foot angle; and
determine a mechanical cycling power associated with the plurality of force sensor readings by inputting the force values, the user mass, the cycling cadence, the foot velocity and the foot angle to a machine learning model trained to predict the mechanical cycling power.

13. The system of claim 12, wherein the plurality of force sensors is disposed on an insole, a shoe, a compression-fit garment, or a sock.

14. The system of claim 12, wherein the one or more processors is further configured to:
output an output dataset, wherein the output dataset comprises the mechanical cycling power and/or the cycling cadence; and
use the output dataset as an input to a game.

15. The system of claim 14, wherein the one or more processors is further configured to generate an audio signal or a visual display based on the output dataset.

16. The system of claim 12, further comprising:
at least one inertial measurement unit associated with the plurality of force sensors, the at least one inertial measurement unit communicatively coupled to the one or more processors;
wherein the one or more processors are configured to determine the cycling cadence based on inertial measurement data received from the at least one inertial measurement unit.

17. The system of claim 12, further comprising:
at least one inertial measurement unit associated with the plurality of force sensors, the at least one inertial measurement unit communicatively coupled to the one or more processors;
wherein the one or more processors are configured to determine the foot angle by:
receiving gyroscope data from the at least one inertial measurement unit; and
determining the foot angle by integrating the gyroscope data in a sagittal plane.

18. The system of claim 12, wherein the one or more processors are configured to determine the foot velocity to include a first foot velocity value in a forward-rearward direction and a second foot velocity value in an upward-downward direction, and to provide the first foot velocity value and the second foot velocity value as separate inputs to the machine learning model.

19. The system of claim 18, wherein the one or more processors are further configured to:
determine the first foot velocity value by receiving accelerometer data from at least one inertial measurement unit associated with the plurality of force sensors and by integrating the accelerometer data in the forward-rearward direction; and
determine the second foot velocity value by receiving accelerometer data from the at least one inertial measurement unit and by integrating the accelerometer data in the upward-downward direction.

20. The system of claim 12, wherein the plurality of force sensors comprise force-sensing resistors and the one or more processors are configured to determine the force values based on a sum of individual sensor force values from the force sensor readings.
